(19) United States
(12) Patent Application Publication
(10) Pub. No.: US 2026/0002107 A1
(43) Pub. Date: Jan. 1, 2026

(54) METHODS TO FABRICATE LIVING CELL VESSELS OF NON-UNIFORM AND SPATIALLY HETEROGENOUS ARCHITECTURE

(71) Applicant: The Texas A&M University System, College Station, TX (US)

(72) Inventors: Abhishek Jain, Cypress, TX (US); Tanmay Mathur, College Station, TX (US); Jennifer Lee, College Station, TX (US); Ankit Kumar, College Station, TX (US)

(21) Appl. No.: 19/256,990

(22) Filed: Jul. 1, 2025

Related U.S. Application Data
(60) Provisional application No. 63/666,622, filed on Jul. 1, 2024.

(51) Int. Cl. C12M 3/06; C12M 1/12; C12N 5/071; C12N 5/077

(52) U.S. Cl. CPC C12M 23/16; C12M 25/14; C12N 5/0661; C12N 5/069; C12N 2533/90

(57) ABSTRACT

A method for fabricating an in vitro vessel. The method may include forming a substrate that defines a microfluidic passage therein including variations in size and shape along a length of the microfluidic passage, positioning the substrate in a vertical orientation whereby an acute angle is formed between a longitudinal direction of extension of the microfluidic passage and a direction of gravity and culturing a plurality of first cells in the microfluidic passage while the substrate is disposed in the vertical orientation whereby an annular layer of the plurality of first cells is formed in the microfluidic passage. The layer of the plurality of first cells may define a lumen extending longitudinally through the microfluidic passage. The lumen includes a complex vascular architecture comprising a bifurcation, tortuosity, a stenosis, an aneurysm, or combinations thereof.

### CROSS-REFERENCE TO RELATED APPLICATIONS

This application claims priority to U.S. Provisional Application Ser. No. 63/666,622 filed on Jul. 1, 2025 and entitled “METHODS TO FABRICATE LIVING CELL VESSELS OF NON-UNIFORM AND SPATIALLY HETEROGENOUS ARCHITECTURE,” which is incorporated herein by reference in its entirety for all purposes.

### STATEMENT REGARDING FEDERALLY SPONSORED RESEARCH OR DEVELOPMENT

This invention was made with government support under Contract No. R01HL157790 awarded by the National Institutes of Health and under Contract No. 1944322 awarded by the Nation Science Foundation. The government has certain rights in the invention.

### TECHNICAL FIELD

The present disclosure relates generally to fabrication of in vitro vessels having non-uniform and spatially heterogeneous architectures. More particularly, the present disclosure relates to methods, systems, and apparatuses for fabricating in vitro vessels with complex architectures pertinent to structures such as stenoses, aneurysms, bifurcations, and tortuous configurations.

### BACKGROUND

The human vascular system exhibits remarkable architectural complexity that extends far beyond simple size variations. For instance, although blood vessels may range from approximately 10 mm in diameter for large arteries to approximately 5 micrometers for small capillaries, the vascular network also displays sophisticated pathological architectures that are critical for understanding disease mechanisms. For example, blood vessels may exhibit complex vascular architectures including heterogeneous features such as stenoses (vessel narrowing), branching patterns at various angles, tortuosity (curved and winding pathways), and aneurysms (localized vessel expansions). Such complex vascular architecture may be most-prominently witnessed in association with cardiovascular complications like atherosclerosis, cerebral aneurysms, arterial tortuosity syndrome, and carotid artery disease, where vessel architecture plays a crucial role in disease onset and progression.

These complex vascular architectures may have functional implications. For example, the herterogenous features may ultimately affect hemodynamics by exerting influence on flow parameters including velocity, flow resistance, pressure, and shear stress. These altered flow behaviors directly affect local mechanobiology and cell morphologies resulting in significant endothelial cell activation and inflammation that subsequently leads to modified cell phenotypes, including activation, coagulation and innate immune responses that are spatially heterogeneous.

In vitro biofabrication techniques, such as Gravitational Lumen Patterning (GLP), have been used to create vessel models useful in studying vessel architecture. GLP enables formation of cylindrical lumens within microfluidic channels. More particularly, GLP utilizes the interplay of surface tension, gravity and fluidic pressure forces between two fluids, having significantly different viscosities, to form a cylindrical lumen within parent microfluidic channels. GLP is based on the physics of “viscous finger patterning,” but incorporates gravitational force to act along the vessel's axial direction during patterning, which prevents the buoyant effect of a relatively lighter fluid being pushed toward the upper surface of the microfluidic channel. The technique involves a fluid dynamics phenomenon where a less viscous fluid, such as a cell medium, flows through and displaces a more viscous liquid, such as liquid collagen, thereby creating structured lumens. However, conventional GLP techniques have been generally limited to the creation of simple, uniform cylindrical vessels embedded in collagen matrices through the use of microfluidic channels with consistent cross-sections throughout their length; however, these uniform cylindrical vessels are generally not capable of accurately modeling pathological architectures such as stenoses, branching patterns at various angles, tortuosity, aneurysms, and the like. A major limitation of the conventional GLP is the lack of spatial relevance that would enable the study of diverse vessels with complex architectures. This limitation significantly restricts the physiological relevance of existing in vitro vessel models for studying cardiovascular diseases, where vessel architecture plays a crucial role in disease onset and progression. There exists no unifying and easy approach to rapidly create the architectural complexity seen in vivo, including the integration of living endothelial cell culture and blood flow. As such, there is a need for methods, systems, and apparatuses for fabricating in vitro vessels with complex architectures pertinent to structures such as stenoses, aneurysms, bifurcations, and tortuous configurations.

### BRIEF SUMMARY OF THE DISCLOSURE

In some embodiments is a method for fabricating an in vitro vessel. The method may comprise forming a substrate that defines a microfluidic passage therein extending along a longitudinal direction of extension and defined by an inner surface. The microfluidic passage may include variations in size and shape along a length of the microfluidic passage. The method may also comprise positioning the substrate in a vertical orientation whereby an acute angle is formed between the longitudinal direction of extension of the microfluidic passage and a direction of gravity. The method may also comprise culturing a plurality of first cells in the microfluidic passage while the substrate is disposed in the vertical orientation whereby an annular layer of the plurality of first cells is formed in the microfluidic passage. The layer of the plurality of first cells may define a lumen extending longitudinally through the microfluidic passage. The lumen may comprise a complex vascular architecture comprising a bifurcation, tortuosity, a stenosis, an aneurysm, or combinations thereof.

Additionally or alternatively, in some embodiments is an in vitro vessel. The in vitro vessel may comprise a substrate forming a microfluidic channel extending along a longitudinal direction of extension and defined by an inner surface, wherein the microfluidic channel extends between a fluid inlet at a first end of the microfluidic channel and a fluid outlet located at a longitudinally opposed second end of the microfluidic channel. The in vitro vessel may also comprise an annular outer layer of muscle cells positioned in the microfluidic channel and extending entirely around the longitudinal direction of extension of the microfluidic channel. The in vitro vessel may also comprise an annular inner layer of endothelial cells positioned within the outer layer of muscle cells within the microfluidic channel and extending entirely around the longitudinal direction of extension of the microfluidic channel. The inner layer of endothelial cells may defines a lumen extending longitudinally through the microfluidic channel and in fluid communication with both the fluid inlet and the fluid outlet formed in the substrate. The lumen may comprise a complex vascular architecture comprising a bifurcation, tortuosity, a stenosis, an aneurysm, or combinations thereof.

Additionally or alternatively, in some embodiments is a method for drug screening. The method may comprise providing an in vitro vessel. The in vitro vessel may comprise a substrate forming a microfluidic channel extending along a longitudinal direction of extension and defined by an inner surface. The microfluidic channel may extend between a fluid inlet at a first end of the microfluidic channel and a fluid outlet located at a longitudinally opposed second end of the microfluidic channel. The in vitro vessel may also comprise an annular outer layer of muscle cells positioned in the microfluidic channel and extending entirely around the longitudinal direction of extension of the microfluidic channel. The in vitro vessel may also comprise an annular inner layer of endothelial cells positioned within the outer layer of muscle cells within the microfluidic channel and extending entirely around the longitudinal direction of extension of the microfluidic channel. The inner layer of endothelial cells may define a lumen extending longitudinally through the microfluidic channel and in fluid communication with both the fluid inlet and the fluid outlet formed in the substrate. The lumen may comprise a bifurcation, tortuosity, a stenosis, an aneurysm, or combinations thereof. The method may further comprise introducing a test compound into the lumen of the in vitro vessel. The method may further comprise perfusing fluid through the lumen to simulate physiological flow conditions. The method may further comprise analyzing endothelial cell responses to the test compound under the complex vascular architecture conditions.

Additionally or alternatively, in some embodiments is a system for modeling vascular disease. The system may comprise an in vitro vessel. The in vitro vessel may comprise a substrate forming a microfluidic channel extending along a longitudinal direction of extension and defined by an inner surface. The microfluidic channel may extend between a fluid inlet at a first end of the microfluidic channel and a fluid outlet located at a longitudinally opposed second end of the microfluidic channel. The in vitro vessel may also comprise an annular outer layer of muscle cells positioned in the microfluidic channel and extending entirely around the longitudinal direction of extension of the microfluidic channel. The in vitro vessel may also comprise an annular inner layer of endothelial cells positioned in the outer layer of muscle cells within the microfluidic channel and extending entirely around the longitudinal direction of extension of the microfluidic channel. The inner layer of endothelial cells may define a lumen extending longitudinally through the microfluidic channel and in fluid communication with both the fluid inlet and the fluid outlet formed in the substrate. The lumen may comprise a bifurcation, tortuosity, a stenosis, an aneurysm, or combinations thereof. The system may comprise a fluid perfusion system configured to establish controlled flow through the lumen. The system may comprise an imaging system configured to monitor cellular responses within the complex vascular architecture.

### BRIEF DESCRIPTION OF THE DRAWINGS

For a detailed description of exemplary embodiments of the disclosure, reference will now be made to the accompanying drawings in which:

FIG. 1 is a schematic illustration of complex vascular structures that mimic pathophysiological anatomies, showing the human vascular system diversity.

FIG. 2 is a schematic view of an embodiment of a stenotic in vitro vessel positioned on a platform.

FIG. 3 is a schematic view of an embodiment of an aneurysmal in vitro vessel positioned on a platform.

FIG. 4 is a schematic view of an embodiment of a bifurcated in vitro vessel positioned on a platform.

FIG. 5 is a schematic view of an embodiment of a tortuous in vitro vessel positioned on a platform.

FIG. 6 is a cross-sectional view of a microfluidic channel showing the annular outer layer of muscle cells and annular inner layer of endothelial cells defining an elliptical lumen.

FIG. 7 is a flowchart of an embodiment of a complex vascular architecture fabrication method.

FIG. 8 is a top and side views of uniform cylindrical vessels formed using GLP with different pressure heads.

FIG. 9 is a graph showing lumen diameter as a function of applied pressure.

FIG. 10 is a top and side views of aneurysm vessels formed in expansion channels.

FIG. 11 is a graph showing maximum widths of aneurysms for different variations.

FIG. 12 is a top and side views of stenotic vessels formed in constriction channels.

FIG. 13 is a graph showing distribution of stenosis widths.

FIG. 14 is a graph showing vessel recovery ratios after stenosis.

FIG. 15 is a graph showing length of stenosis sections.

FIG. 16 is a top and side views of bifurcating vessels in different angle channels.

FIG. 17 is a linear regression analysis between bifurcation angles of pre-GLP and post-GLP lumens.

FIG. 18 is a graph showing pre-bifurcation lumen diameters.

FIG. 19 is a graph showing post-bifurcation lumen diameters.

FIG. 20 is a top and side views of tortuous vessels in different peak-to-peak channels.

FIG. 21 is a graph showing lumen tortuosity index measurements.

FIG. 22 is a linear regression analysis between tortuosity index of pre-GLP and post-GLP tortuous vessels.

FIG. 23 is a fluorescence confocal micrograph of endothelial cells cultured in cylindrical vessel-chip.

FIG. 24 is a polar plot showing endothelial cell orientation analysis in cylindrical vessels.

FIG. 25 is a fluorescence confocal micrograph of endothelial cells cultured in aneurysm vessel-chip.

FIG. 26 is a polar plot showing endothelial cell orientation in pre-aneurysm sections.

FIG. 27 is a polar plot showing endothelial cell orientation in intra-aneurysm sections.

FIG. 28 is a polar plot showing endothelial cell orientation in post-aneurysm sections.

FIG. 29 is a graph showing cell shape index analysis in aneurysm regions.

FIG. 30 is a fluorescence confocal micrograph of endothelial cells cultured in stenotic vessel-chip.

FIG. 31 is a polar plot showing endothelial cell orientation in pre-stenosis sections.

FIG. 32 is a polar plot showing endothelial cell orientation in stenosis sections.

FIG. 33 is a polar plot showing endothelial cell orientation in post-stenosis sections.

FIG. 34 is a graph showing cell shape index analysis in stenosis regions.

FIG. 35 is a fluorescence confocal micrograph of endothelial cells cultured in bifurcation vessel-chip.

FIG. 36 is a polar plot showing endothelial cell orientation in pre-bifurcation sections.

FIG. 37 is a polar plot showing endothelial cell orientation in bifurcation sections.

FIG. 38 is a polar plot showing endothelial cell orientation in post-bifurcation sections.

FIG. 39 is a graph showing cell shape index analysis in bifurcation regions.

FIG. 40 is a fluorescence confocal micrograph of endothelial cells cultured in tortuous vessel-chip.

FIG. 41 is a polar plot showing endothelial cell orientation in pre-tortuous sections.

FIG. 42 is a polar plot showing endothelial cell orientation in tortuous sections.

FIG. 43 is a polar plot showing endothelial cell orientation in post-tortuous sections.

FIG. 44 is a graph showing cell shape index analysis in tortuous regions.

FIG. 45 is brightfield micrographs showing red blood cell flow in cylindrical vessels.

FIG. 46 is a time averaged wall shear stress distribution heatmap for cylindrical vessels.

FIG. 47 is a graph comparing normalized TAWSS between different vessel diameters.

FIG. 48 is a graph showing normalized residence time for different vessel diameters.

FIG. 49 is brightfield micrographs showing red blood cell flow in aneurysm vessels.

FIG. 50 is a time averaged wall shear stress distribution heatmap for aneurysm vessels.

FIG. 51 is a graph comparing normalized TAWSS between different aneurysm widths.

FIG. 52 is a graph showing normalized residence time for different aneurysm widths.

FIG. 53 is brightfield micrographs showing red blood cell flow in stenotic vessels.

FIG. 54 is a time averaged wall shear stress distribution heatmap for stenotic vessels.

FIG. 55 is a graph comparing normalized TAWSS between different stenosis percentages.

FIG. 56 is a graph showing normalized residence time for different stenosis percentages.

FIG. 57 is brightfield micrographs showing red blood cell flow in bifurcation vessels.

FIG. 58 is a time averaged wall shear stress distribution heatmap for bifurcation vessels.

FIG. 59 is a graph comparing normalized TAWSS between different bifurcation angles.

FIG. 60 is brightfield micrographs showing red blood cell flow in tortuous vessels.

FIG. 61 is a time averaged wall shear stress distribution heatmap for tortuous vessels.

FIG. 62 is a graph comparing normalized TAWSS between different tortuosity indices.

FIG. 63 is a graph showing normalized residence time for different tortuosity indices.

### DETAILED DESCRIPTION OF THE DISCLOSED EMBODIMENTS

The following discussion is directed to various exemplary embodiments. However, one skilled in the art will understand that the examples and embodiments disclosed herein have broad application, and that the discussion of any embodiment is meant only to be exemplary of that embodiment, and not intended to suggest that the scope of the disclosure, including the claims, is limited to that embodiment.

Certain terms are used throughout the following description and claims to refer to particular features or components. As one skilled in the art will appreciate, different persons may refer to the same feature or component by different names. This document does not intend to distinguish between components or features that differ in name but not function. The drawing figures are not necessarily to scale. Certain features and components herein may be shown exaggerated in scale or in somewhat schematic form and some details of conventional elements may not be shown in interest of clarity and conciseness.

In the following discussion and in the claims, the terms “including” and “comprising” are used in an open-ended fashion, and thus should be interpreted to mean “including, but not limited to . . . ” Also, the term “couple” or “couples” is intended to mean either an indirect or direct connection. Thus, if a first device couples to a second device, that connection may be through a direct connection, or through an indirect connection via other devices, components, and connections. In addition, as used herein, the terms “axial” and “axially” generally mean along or parallel to a central axis (e.g., the central axis of a body or a port), while the terms “radial” and “radially” generally mean perpendicular to the central axis. For instance, an axial distance refers to a distance measured along or parallel to the central axis, and a radial distance means a distance measured perpendicular to the central axis.

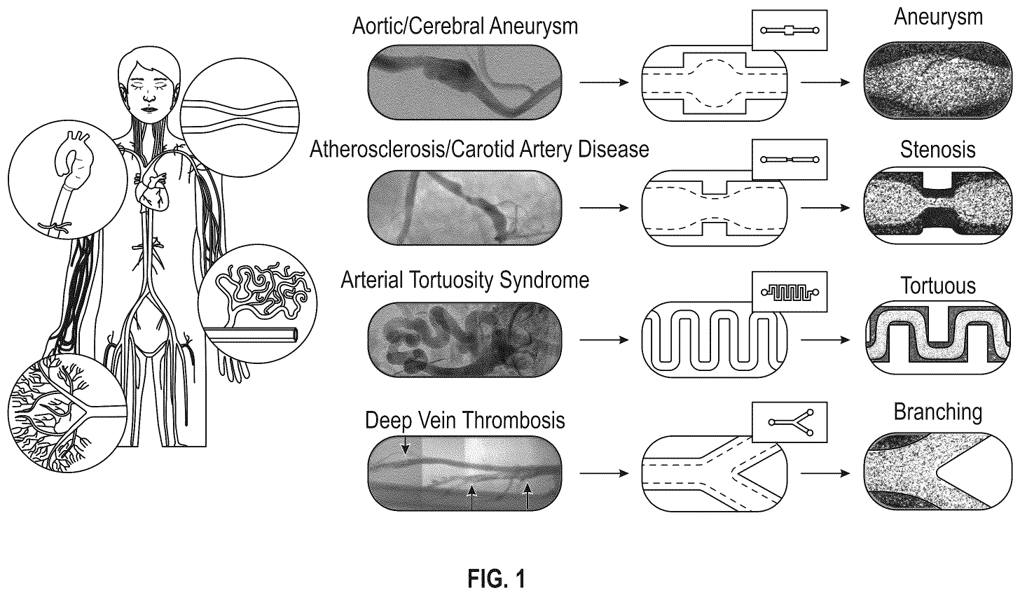

Disclosed herein are various embodiments of the present disclosure relates to methods, systems, and apparatuses for fabricating in vitro vessels with complex architectures pertinent to structures such as stenoses, aneurysms, bifurcations, and tortuous configurations. As used herein, the term “complex vascular architecture” refers to in vitro vessel structures that exhibit non-uniform geometric configurations along a longitudinal direction of extension. FIG. 1 illustrates examples of such complex vascular architectures observed in vivo, including cerebral aneurysms, stenotic regions, arterial tortuosity, and thrombotic formations, alongside the corresponding engineered structures achievable through the disclosed GLP-based fabrication methods. Also, as used here, the term “longitudinal direction of extension” refers to the generally direction in which in vitro vessel extends within a substrate between an inlet to the in vitro vessel and an out of the in vitro vessel, regardless of any deviation along the vessel. For example, a vessel exhibiting complex vascular architecture may be characterized by spatial variations in lumen diameter, variations in cross-sectional shape, variations in directional orientation, or branching patterns that deviate from simple cylindrical or uniform tubular geometries. Features present in a complex vascular architectures may include, but are not limited to, stenotic regions having constricted lumen diameters with varying degrees of closure (for example, exhibiting a lumen cross-section of less than 75%, 50%, or 25% of a non-stenoic region), aneurysmal regions characterized by localized expansions or enlargements of the vessel lumen (for example, exhibiting a lumen cross-section of at least 150%, 200%, 300%, or 400% of a non-aneurysmal region), bifurcated structures where a single vessel branches into multiple branches at various angles (for example, where two branches deviate and extend at an angle of about 30°, 40°, 50°, 60°, 70°, 80°, or) 90°, tortuous configurations exhibiting curved or winding pathways exhibiting multiple changes in direction and/or exhibiting peak-to-peak height variations, or combinations thereof. Generally, the features present in such complex vascular architectures may mimic heterogeneous vessel geometries and/or other features observed in vivo under both physiological and pathological conditions, including those associated with cardiovascular diseases such as atherosclerosis, arterial tortuosity syndrome, aortic and cerebral aneurysms, and carotid artery disease.

As will be disclosed herein, the disclosed the complex vascular architecture fabrication methods, systems, and apparatuses may utilize a GLP-based technique to generate one or more features associated with complex vascular architecture that were previously not achievable using existing biofabrication techniques. More particularly, and as will be disclosed herein, the complex architectures may be formed through controlled manipulation of the geometries microfluidic channel in which the vessel is formed and that dictate the spatial patterning of the embedded vessel lumen during fabrication using GLP. For example, in some embodiments, the complex vascular architecture fabrication methods, systems, and apparatuses disclosed herein generally utilize non-uniform parent microfluidic channels having particular geometries, as will be disclosed herein, to dictate lumen patterning. Generally, a “parent microfluidic channel” refers to a microfluidic channel in which lumen, more particularly, an in vitro vessel, is formed. For example, the lumens formed according to the complex vascular architecture fabrication methods, systems, and apparatuses disclosed herein may be governed by the shape and size of the parent microfluidic channels used to pattern the vessels having complex architecture to create complex lumens with varying shapes and sizes through the local changes in pressure experienced by the two different fluids used to induce viscous fingering.

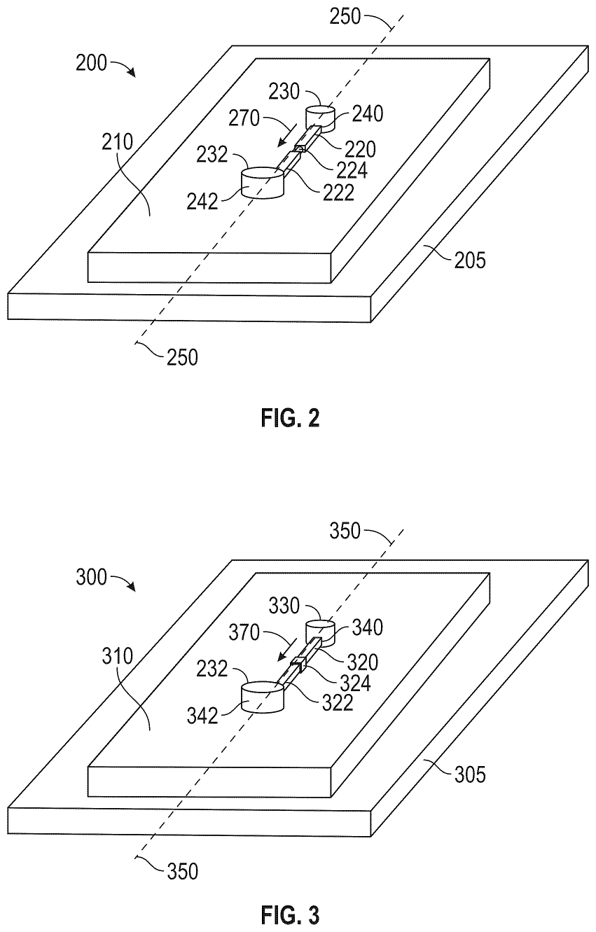

Referring now to FIG. 2, an embodiment of a stenotic in vitro vessel 200 is shown. The in vitro vessel 200 generally includes a substrate 210 positioned on a platform 205. The substrate 210 comprises and at least partially defines a microfluidic channel 220 therein that extends along a longitudinal direction of extension 250. The microfluidic channel is defined by an inner surface 222. The microfluidic channel 220 includes one or more stenotic region(s) 224 characterized by constricted lumen diameters, creating variations in cross-sectional area along the length of the vessel. In this exemplary embodiment, microfluidic channel 220 extends from a first or inlet end 230 to a second or outlet end 232 longitudinally opposite the inlet end 230, with stenotic constrictions positioned at predetermined locations along the length of the microfluidic channel. In addition to microfluidic channel 220, the substrate also comprises a fluid inlet 240 and a fluid outlet 242 such that a fluid flow 270 may be established through the microfluidic channel 220 and the stenotic region(s) 224.

Referring to FIG. 3, an embodiment of an aneurysmal in vitro vessel 300 is shown. The in vitro vessel 300 generally includes a substrate 310 positioned on a platform 305.

The substrate 310 comprises and at least partially defines a microfluidic channel 320 therein that extends along a longitudinal direction of extension 350. The microfluidic channel is defined by an inner surface 322. The microfluidic channel 320 includes one or more aneurysmal region(s) 324 characterized by localized expansions or enlargements of the vessel lumen, creating variations in cross-sectional area along the length of the vessel. In this exemplary embodiment, microfluidic channel 320 extends from a first or inlet end 330 to a second or outlet end 332 longitudinally opposite the inlet end 330, with aneurysmal expansions positioned at predetermined locations along the length of the microfluidic channel. In addition to microfluidic channel 320, the substrate also comprises a fluid inlet 340 and a fluid outlet 342 such that a fluid flow 370 may be established through the microfluidic channel 320 and the aneurysmal region(s) 324.

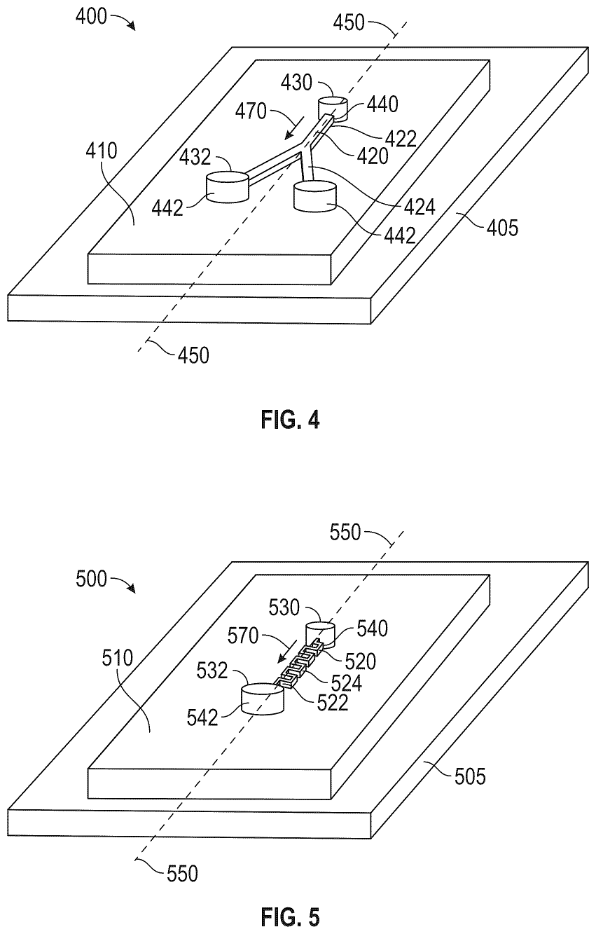

Referring to FIG. 4, an embodiment of a bifurcated in vitro vessel 400 is shown. The in vitro vessel 400 generally includes a substrate 410 positioned on a platform 405. The substrate 410 comprises and at least partially defines a microfluidic channel 420 therein that extends along a longitudinal direction of extension 450. The microfluidic channel is defined by an inner surface 422. The microfluidic channel 420 includes a bifurcated structure 424 where a single vessel branches into multiple branches at an angle, creating a deviation in directional orientation along the length of the vessel. In this exemplary embodiment, microfluidic channel 420 extends from a first or inlet end 430 and branches into two or more outlet ends 432 positioned at predetermined angles. In addition to microfluidic channel 420, the substrate also comprises a fluid inlet 440 and multiple fluid outlets 442 such that a fluid flow 470 may be established through the microfluidic channel 420 and the bifurcation structure 424.

Referring to FIG. 5, an embodiment of a tortuous in vitro vessel 500 is shown. The in vitro vessel 500 generally includes a substrate 510 positioned on a platform 505. The substrate 510 comprises and at least partially defines a microfluidic channel 520 therein that extends along a longitudinal direction of extension 550. The microfluidic channel is defined by an inner surface 522. The microfluidic channel 520 includes tortuous configuration(s) 524 exhibiting curved or winding pathways with multiple changes in direction and peak-to-peak height variations, creating variations in directional orientation along the length of the vessel. In this exemplary embodiment, microfluidic channel 520 extends from a first or inlet end 530 to a second or outlet end 532 longitudinally opposite the inlet end 530, with tortuous features positioned at predetermined locations along the length of the microfluidic channel. In addition to microfluidic channel 520, the substrate also comprises a fluid inlet 540 and a fluid outlet 542 such that a fluid flow 570 may be established through the microfluidic channel 520 and the tortuous configuration(s) 524.

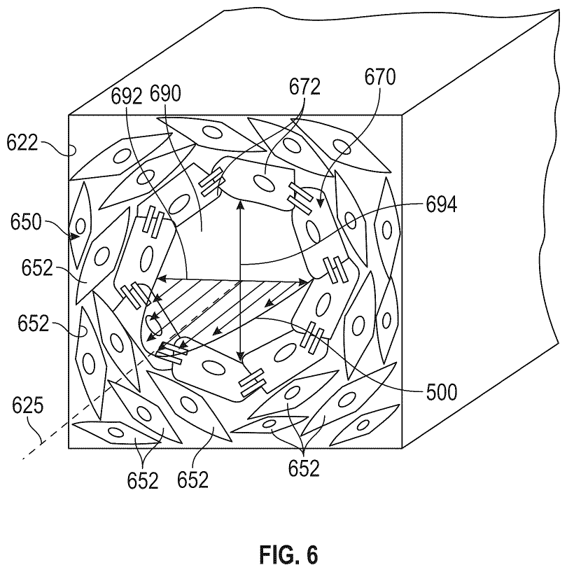

Referring to FIG. 6, a cross-section of the microfluidic channel (such as the microfluid channel 200, 300, 400, of 500 of FIGS. 2-5) an in vitro vessel having complex vascular architecture is shown. In the embodiment of FIG. 6, an annular first or outer layer 650 is formed in the microfluidic channel and extending longitudinally between the inlet end and the outlet end of the microfluidic channel. In this exemplary embodiment, the outer layer 650 extends continuously and entirely (360°) around the longitudinal direction of extension 625 of the microfluidic channel. Additionally, an annular second or inner layer 670 is also formed in the microfluidic channel and extending longitudinally between the inlet end and the outlet end of the microfluidic channel. The inner layer 670 defines a fluid passage or the lumen 690 through which fluid flow extends and which is in fluid communication with both the fluid inlet and the fluid outlet formed in the substrate. In this configuration, the lumen 690 extends longitudinally through the microfluidic channel such that the longitudinal direction of extension 625 extends longitudinally through the lumen 690. Additionally, the inner layer 670 extends continuously and entirely (360°) around the longitudinal direction of extension 625 of the microfluidic channel. In this configuration, the outer layer 650 is positioned radially between the inner layer 670 and the inner surface 622 of the microfluidic channel whereby the outer layer 650 entirely surrounds or encircles the inner layer 670 positioned therein. In this exemplary embodiment, the lumen 690 formed by the inner layer 670 has an elliptical cross-section defined by a first or major axis 692 and a second or minor axis 694 extending orthogonal to the major axis 692, and where each axis 692 and 694 intersects the longitudinal direction of extension 625 of the microfluidic channel. The elliptical cross-section of lumen 690 is more accurately reflects in vivo vessels, such as human lymphatic and vascular vessels, than the lumen formed by conventional in vitro vessels which may instead have a rectangular or square cross-section. Particularly, the elliptical geometry of the lumen 690 permits the in vitro vessel to better mimic the physiological conditions (e.g., physiological stress and strain conditions) found in in vivo vessels. In this exemplary embodiment, the outer layer 650 formed in the microfluidic channel comprises a plurality of first cells 652 while the inner layer 670 formed in the microfluidic channel and surrounded by the outer layer 650 comprises a plurality of second cells 672 which are different from the first cells 652. The first cells 652 and second cells 672 may be cultured using a GLP fabrication process, as will be disclosed herein. Generally, the first cells 652 and second cells 672 are each distributed substantially uniformly circumferentially about the longitudinal direction of extension 625 of the microfluidic channel such that the cells 652 and/or 672 do not collect or congregate at one circumferential location along the circumference of the microfluidic channel. In this manner, while the radial thickness of each layer 650 and 670 may vary to some degree moving circumferentially along the layer 650 and 670, the variation in radial thickness of each layer 650 and 670 moving circumferentially along the layer 650 and 670 is not particularly significant. For example, in some embodiments, the ratio of the minimum radial thickness of each layer 650 and 670 to the maximum radial thickness of each layer 650 and 670 is approximately between 0.5:1 and 2:1.

The first cells 652 and second cells 672 may each have a specific alignment relative to the direction of the longitudinal direction of extension 625 of the microfluidic channel. For example, in some embodiments, a majority of the first cells 652 comprising the outer layer 650 align in a direction that is substantially perpendicular to the longitudinal direction of extension 625 of the microfluidic channel while a majority of the second cells 672 forming the inner layer 670 align in a direction that is substantially parallel to the longitudinal direction of extension 625. In this manner, the first cells 652 of outer layer 650 wrap circumferentially around the axially-aligned second cells 672 which align in the direction of fluid flow through the lumen 690. As described above, the first cells 652 are different from the second cells 672. Particularly, in this exemplary embodiment, the first cells 652 comprise mural or muscle cells while the second cells 672 comprise endothelial cells. In some embodiments, the in vitro vessel comprises a physiologically relevant lymphatic in vitro vessel with muscle cells 652 comprising LMCs and endothelial cells 672 comprising LECs. In other embodiments, the in vitro vessel comprises a physiologically relevant vascular or blood in vitro vessel with muscle cells 652 comprising VMCs and endothelial cells 672 comprising VECs. This cellular organization and lumen formation is consistent across all complex vascular architectures disclosed herein, including stenotic regions, aneurysmal regions, bifurcation structures, and tortuous configurations.

The following detailed description explains embodiments of fabrication methodologies for creating each type of complex vessel architecture as disclosed herein. While the fundamental cellular organization illustrated in FIG. 6 remains consistent across stenotic, aneurysmal, bifurcated, and tortuous vessel configurations, the formation of these distinct architectural features is achieved through controlled variations in the microfluidic channel geometries and specific modifications to the GLP fabrication process. As will be described in detail below, the shape and size of the pre-GLP microfluidic channels dictate the spatial patterning of the embedded vessel lumen during fabrication, enabling the creation of physiologically relevant complex vascular architectures while preserving the uniform cellular distribution and elliptical lumen geometry described above. The following methodology sections will detail how non-uniform external microfluidic geometries are utilized to create the local pressure variations necessary for forming each specific type of complex vascular architecture through the GLP biofabrication technique

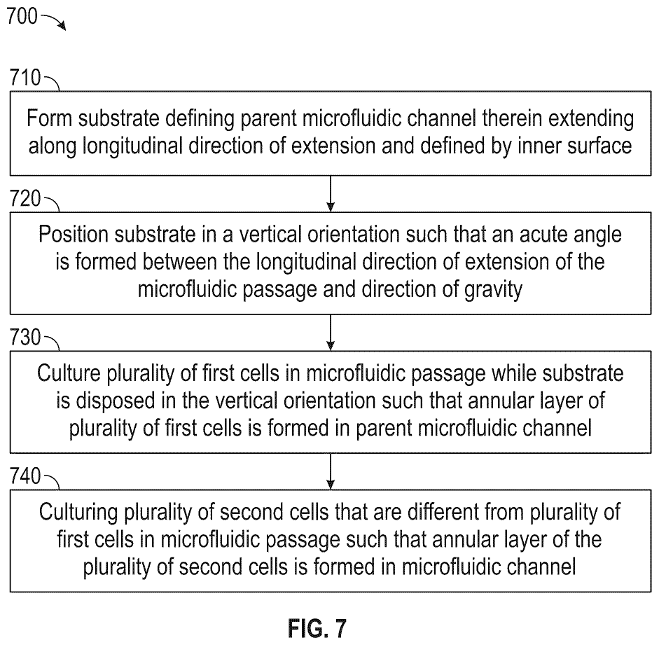

In some embodiments, for example, referring to FIG. 7, a complex vascular architecture fabrication method 700 may generally comprise forming the substrate defining a parent microfluidic channel therein extending along a longitudinal direction of extension and defined by an inner surface 710. The parent microfluidic passage may include variations in size and shape along its length. The complex vascular architecture fabrication method 700 may further comprise positioning the substrate in a vertical orientation such that an acute angle is formed between the longitudinal direction of extension of the microfluidic passage and a direction of gravity 720. The complex vascular architecture fabrication method 700 may further comprise culturing a plurality of first cells in the microfluidic passage while the substrate is disposed in the vertical orientation such that an annular layer of the plurality of first cells is formed in the parent microfluidic channel 730. The layer of the plurality of first cells, for example, endothelial cells, may define a lumen extending longitudinally through the parent microfluidic channel and having a complex architecture, for example, comprising a feature such as a bifurcation, tortuosity, a stenosis, or an aneurysm. As such, the disclosed complex vascular architecture fabrication method may enable creation of living three-dimensional (3D) and closed vascular lumens embedded in a collagen matrix and lined with endothelial cells. Additionally, in some embodiments, the complex vascular architecture fabrication method 700 may further comprise culturing a plurality of second cells that are different from plurality of first cells in the microfluidic passage such that an annular layer of the plurality of second cells is formed in the microfluidic channel 740.

In some embodiments, the substrate formation process generally comprises designing parent microfluidic channels, generally configured to yield the complex vascular architecture, for example, including a feature such as bifurcations, tortuosity, stenoses, and aneurysms, when vessels are formed via a GLP technique. For each type of vessel feature, the particular parameters of the feature may be modulated by varying one or more specific geometric parameters such as the angle between bifurcated channels, levels of tortuosity, degrees of stenosis, degree of expansion, or combinations thereof.

In some embodiments, a parent microfluidic channel may be configured to yield a bifurcated vessel and may comprise a channel having a cross-sectional area generally perpendicular to a direction of extension of from about 400 micrometers to about 600 micrometers, for example, about 500 micrometers, by from about 400 micrometers to about 600 micrometers, for example, about 500 micrometers that branches into two channels having a cross-sectional area generally perpendicular to a direction of extension of from about 250 micrometers to about 450 micrometers, for example, about 350 micrometers by from about 400 micrometers to about 600 micrometers, for example, about 500 micrometers channels. In various embodiments, an angle between the two bifurcated channels may be about 30°, 40°, 50°, 60°, 70°, 80°, or 90°. In various embodiments, the bifurcated channel design generally comprises a single channel of about 3 millimeters to about 7 millimeters, for example, about 5 millimeters in length that branches into two channels of about 3 millimeters to about 7 millimeters, for example, about 5 millimeters each.

Additionally or alternatively, in some embodiments, a parent microfluidic channel may be configured to yield one or more tortuous vessels and may comprise a channel having a cross-sectional area generally perpendicular to a direction of extension of from about 400 micrometers to about 600 micrometers, for example, about 500 micrometers by from about 400 micrometers to about 600 micrometers, for example, about 500 micrometers. The parent microfluidic channel configured to yield a tortuous vessel may exhibit a generally rectangular waveform, for example, including from about two (2) to about twelve (12) cycles, for example, about eight cycles of rectangular waveform with about 2 millimeters of length per cycle. The parent microfluidic channel(s) may extend generally straight over a length and then may deviate, such that the channel has a peak-to-peak heights from about 1 millimeter to about 5 millimeters, for example, of about 1.5 millimeters, 2 millimeters, and/or 3 millimeters. The parent microfluidic channel may have a cross-sectional area generally perpendicular to a direction of extension of from about 400 micrometers to about 600 micrometers, for example, about 500 micrometers by from about 400 micrometers to about 600 micrometers, for example, about 500 micrometers a tortuous region of the parent microfluidic channel. In some embodiments, the parent microfluidic channel may include a length, for example, of about 5 millimeters length, of straight channels before and after the tortuous region to provide entrance length for flow stabilization.

Additionally or alternatively, in some embodiments, a parent microfluidic channel may be configured to yield one or more stenotic vessels and may comprise a channel having a cross-sectional area generally perpendicular to a direction of extension of from about 400 micrometers to about 600 micrometers, for example, about 500 micrometers, by from about 400 micrometers to about 600 micrometers, for example, about 500 micrometers. The parent microfluidic channel configured to yield a stenotic vessel may contain from about one (1) to about five (5) spaced constrictions, for example, equally-spaced, for example, about three equally-spaced constrictions. In various embodiments, the each of the constrictions may have one a cross-sectional area that is from about 20% to about 80% of the cross-sectional area of a non-constricted portion of the parent microfluidic channel, for example, a cross-sectional area of about 25%, about 50%, or about 75% of the cross-sectional area of the non-constricted portion of the parent microfluidic channel. The geometry associated with each constriction (e.g., which will yield a stenosis) may be generally rectangular, having an about one-to-one ratio for the length and width. In some embodiments, a single stenosed channel may have a total channel length of from about 15 millimeters to about 25 millimeters, for example, about 20 millimeters. The cross-sectional area generally perpendicular to a direction of extension may remain from about 400 micrometers to about 600 micrometers, for example, about 500 micrometers by from about 400 micrometers to about 600 micrometers, for example, about 500 micrometers excluding the stenosed regions.

Additionally or alternatively, in some embodiments, a parent microfluidic channel may be configured to yield one or more aneurysmal vessels and may comprise a channel having a cross-sectional area generally perpendicular to a direction of extension of from about 400 micrometers to about 600 micrometers, for example, about 500 micrometers by from about 400 micrometers to about 600 micrometers, for example, about 500 micrometers. The parent microfluidic channel configured to yield an aneurysmal vessel may contain from about one (1) to about three (3) spaced expansions, for example, equally-spaced, for example, about two equally-spaced expansions. In various embodiments, each of the expansions may have a cross-sectional area that is from about 150% to about 500% of the cross-sectional area of a non-expanded portion of the parent microfluidic channel, for example, a cross-sectional area of about 200% or about 400% of the cross-sectional area of the non-expanded portion of the parent microfluidic channel. The geometry associated with each expansion (e.g., which will yield an aneurysm) may be generally rectangular, and its length and width may be from about 0.5 millimeters to about 3 millimeters, for example, about 1 millimeter or about 2 millimeters based on the desired level of expansion. In some embodiments, a single aneurysmal channel may have a total channel length of from about 15 millimeters to about 25 millimeters, for example, about 20 millimeters. The cross-sectional area generally perpendicular to a direction of extension may remain from about 400 micrometers to about 600 micrometers, for example, about 500 micrometers by from about 400 micrometers to about 600 micrometers, for example, about 500 micrometers excluding the aneurysmal regions.

In some embodiments, the substrate formation process also generally comprises fabricating the substrate based upon an advanced manufacturing techniques suitable for creating non-uniform parent microfluidic channel geometries. For example, in some embodiments, the substrate is formed via a soft lithography process. This manufacturing approach enables precise control over the complex geometries needed for complex vascular architectures while maintaining dimensional accuracy throughout the fabrication process.

In some embodiments, the soft lithography process may comprise forming a mold from which the substrate may be formed.

In some embodiments, the substrate formation process also generally comprises creating one or more substrate molds in which the substrate will be formed. The substrate molds may function as an intermediate manufacturing template and may define the specific geometric variations defining the parent microfluidic channels. For example, the substrate molds may function as negative templates.

In some embodiments, the parent microfluidic channels that are configured to yield the complex vascular architecture may be incorporated into the design for the mold. For example, the 3D design for each mold may be created using suitable design software, an example of which is commercially available as Autodesk Fusion 360™. The 3D design(s) may incorporate the specific parent microfluidic channels, each having one or more geometric variations to yield the complex vessel architecture. In some embodiments, all parent microfluidic channels are designed to have an end-to-end length of about 2 centimeters, for example, such that the parent microfluidic channels may be contained entirely within a mold of a particular size. For example, the thickness of the base and walls of the mold may be about 5 millimeters, and the total dimension of the mold is may be about 21 millimeters by about 50 millimeters by about 10 millimeters.

The substrate mold may then be formed. For example, the design for the mold may be exported, for example, as an STL file, and may be printed using suitable 3D printing equipment, as example, of which is commercially available as the Asiga Max stereolithography (SLA) printer. The substrate molds may be 3D printed using a suitable material, for example, a clear resin such as (meth)acrylates, an example, of which is commercially available as audioprint® GR-10. In some embodiments, after printing, the substrate mold may be sonicated in about 100% isopropyl alcohol (IPA) for a duration sufficient to remove excess uncured resin, for example, about 30 minutes. After removal of excess uncured resin, the substrate mold may be subjected to UV treatment in an inert environment, for example, nitrogen gas (N₂) to complete curing of the resin.

In some embodiments, following mold production, substrate fabrication generally comprises forming the substrate, for example, by patterning substrate mold via standard soft lithography methods. Soft lithography is a microfabrication technique used to create microfluidic devices from a suitable material, for example, an elastomeric material such as polydimethylsiloxane (PDMS), a polyurethane, a polyamide, a cross-linked Novolac resin, a hydrogel, a polyolefin, perfluoropolyether (PFPE), or combinations thereof. Additionally or alternatively, other materials such as silicon

In some embodiments, the soft lithography process begins by mixing PDMS prepolymers with a curing agent in about a 10:1 ratio and pouring the prepared PDMS mixtures into the molds, followed by degassing in a vacuum desiccator to remove traces of air. During this process, the PDMS takes on the shape defined by the mold, enabling the translation of the 3D-printed geometries incorporated into the substrate mold into functional PDMS substrates, more particularly, the PDMS takes on the shape of the parent microfluidic channels.

In some embodiments, one or more substrate molds containing PDMS are then placed in an oven at about 70° C. for at least about two (2) hours to complete the curing process. During this time, the PDMS polymerizes and solidifies, taking on the shape defined by the mold. Once the polymerization process is completed, PDMS layers or “stamps” are carefully removed from the molds to form the substrate defining the parent microfluidic channel. Inlet and outlet ports may be created at the ends of each channel using suitable tools such as a 1-millimeter biopsy punch (Miltex-Integra), and excess PDMS is trimmed to leave sufficient surface area for bonding to enclose the channel.

In some embodiments, the substrate may be bonded to a thin glass slide, which may serve to define the fourth side of the parent microfluidic channels. The thin glass slide may be pre-coated with PDMS such that all inner surfaces of the parent microfluidic channels comprise the same material. The molded PDMS pieces and PDMS-coated glass slides are oxygen plasma treated using about 75 watts oxygen plasma at about 0.80 mbar for about 30 seconds so as to be chemically bonded to each other. Once bonded, devices are stored in an oven at about 70° C. overnight under weighted conditions ranging from about 100 grams to about 500 grams disposed on top of each device to complete the bonding process, resulting in irreversibly bonded, completely sealed channels containing the complex geometric variations originally designed into the 3D printed molds.

Alternatively, in some embodiments, microfluidic channels as disclosed herein may be created within a glass or plastic materials.

In some embodiments, the complex vascular architecture fabrication method comprises positioning the substrate in a vertical orientation such that an acute angle is formed between the longitudinal direction of extension of the parent microfluidic channel and a direction of gravity. Generally, positioning the substrate in the vertical orientation generally comprises rotating the substrate by approximately 90° from a horizontal position to align or substantially align the longitudinal axis of the parent microfluidic channel with the gravitational direction. Not intending to be bound by theory, this position of the substrate allows gravitational force to act along the longitudinal direction of extension of the parent microfluidic channel during formation of the in vitro vessels, which prevents the buoyant effect of a relatively less dense fluid being pushed toward the upper surface of the microfluidic channel. The vertical orientation mitigates the buoyancy effects that might otherwise impair the formation of the lumen by ensuring that the force of gravity acts on the cells cultured therein in a direction parallel to the direction of extension of the parent microfluidic channel. In some embodiments, the vertical orientation of the substrate generally enables the cells cultured in the parent microfluidic channel to achieve a uniform or substantially uniform distribution along the inner surface of the parent microfluidic channel. Because the substrate is oriented vertically, cells are not drawn from the top of the parent microfluidic channel towards the bottom of the parent microfluidic channel, thereby preventing the formation of asymmetrical cell layers that would be thick at the bottom and thin at the top. This uniform distribution may yield more physiologically relevant vessel models that accurately mimic the structural characteristics observed in vivo.

In some embodiments, the angle, for example, an acute angle, formed between the longitudinal direction of extension of the parent microfluidic channel and the direction of gravity (e.g., vertical) may be equal to or less than about 30°, additionally or alternatively, less than about 20°, about 15°, about 10°, about 5°, or about 1°. In certain embodiments, the acute angle may be zero with respect to the direction of gravity, for example, with the substrate oriented vertically.

In some embodiments, the substrate may be positioned in the vertical orientation after the parent microfluidic channel has been filled with an extracellular matrix material but before the introduction of a less viscous fluid that will create the lumen, as will be disclosed herein. This timing may help to ensure that the gravitational effects are effectively applied during the lumen formation phase of the GLP technique, as will be disclosed herein.

In some embodiments, the complex vascular architecture fabrication method comprises preparing the parent microfluidic channel for subsequent gravitational lumen patterning and cell culturing processes through chemical pretreatment. The parent microfluidic channels may be chemically pretreated to enhance bonding of the PDMS surfaces of the parent microfluidic channels and an extracellular matrix (ECM). The parent microfluidic channels may first be oxygen plasma treated to activate the PDMS surfaces. Additionally, the parent microfluidic channel may then be treated with a functionalization agent such as about 10% v/v 3-aminopropyltriethoxysilane (APTES) in about 200 proof ethanol, which functions as a silane coupling agent that creates chemical bonds between the PDMS substrate and the subsequently applied collagen matrix. The channels are incubated for about 15 minutes at room temperature before thorough successively rinsing with about 100% and about 70% ethanol solutions, respectively. The channels may then be completely dried and then incubated in an oven at about 70° C. for about 30 minutes. The channels are then injected with a cross-linking agent such as about a 2.5% v/v solution of glutaraldehyde in phosphate buffered saline (PBS), which serves as a cross-linking agent to further strengthen the bonds between the ECM and PDMS surfaces, followed by incubation at room temperature for about 30 minutes, then extensively washed with about 100% and about 70% ethanol solutions to remove any traces of glutaraldehyde. This chemical pretreatment process ensures structural integrity of the complex vascular architectures during the gravitational lumen patterning process and subsequent cell culturing steps.

In some embodiments, following chemical pretreatment, the complex vascular architecture fabrication method comprises performing gravitational lumen patterning to create the lumen within the parent microfluidic channel. A collagen solution may be prepared according to manufacturer's protocol, with the collagen stock reconstituted to a concentration in a range of about 5 mg/mL to about 7.5 mg/mL, with the pH adjusted to about 7.5 using sodium bicarbonate, sodium hydroxide, and 4-(2-hydroxyethyl)-1-piperazineethanesulfonic acid (HEPES). The pH adjustment to 7.5 may be effective to allow for proper collagen polymerization, as collagen fibrillogenesis is highly pH-dependent and occurs optimally at physiological pH levels, with acidic conditions preventing proper cross-linking and alkaline conditions potentially denaturing the collagen structure. The combination of sodium bicarbonate (7.5%), sodium hydroxide, and HEPES (1 M) may be effective to provide precise pH control and buffering capacity to maintain stable conditions during the subsequent polymerization process. Generally, the concentration of collagen is directly proportional to both the viscosity of the collagen solution and the hydraulic resistance of the collagen solution. As such, as will be disclosed herein, changes in the concentration of collagen may be effective to yield manipulate lumen size during the GLP process. Collagen mixtures are prepared cold to prevent premature cross-linking of collagen solutions, which would interfere with the subsequent viscous fingering process that creates the lumen. Prior to injecting collagen solutions into the parent microfluidic channel, pipette tips, such as 200 μL pipette tips, are bent to about 135° about 7 mm from the tip and pushed into the inlet and outlet ports of the parent microfluidic channel to facilitate proper fluid flow during the patterning process. Using a pipette, cold collagen is injected into the parent microfluidic channel until the levels of collagen are roughly equal between the inlet and outlet, and collagen is allowed to equilibrate for about 15 seconds to allow the filled collagen to stabilize and reach equilibrium before the vertical positioning step.

In some embodiments, after filling the parent microfluidic channel with the collagen solution, the substrate may then be positioned in the vertical orientation as previously disclosed. With the substrate in the vertical orientation, PBS, for example, about 40 μL of 1× PBS, may be introduced through a bent tip connected to the inlet. Generally, the gravitational lumen patterning process relies on the fundamental physics of viscous fingering, which is a fluid dynamics phenomenon in which a less viscous fluid flows through and displaces a more viscous liquid, creating finger-shaped features. The vertical orientation of the substrate allows gravitational force to act in the axial direction of the channel, which may provide control over buoyancy effects that may influence lumen formation. Specifically, by maintaining the substrate vertically, thereby aligning the gravitational force in the longitudinal direction of extension of the parent microfluidic channel, a 3D lumen having a nearly symmetrical cross-section can be obtained. During this process, the less viscous fluid, for example, the PBS, may tunnel through the more viscous fluid, for example, the collagen solution, through viscous fingering. The substrates may be incubated at about 37° C. in a humidified environment of about 5% CO₂ while maintaining the substrate vertically and incubated for a sufficient time for the less viscous fluid to displace the highly viscous fluid and form a 3D lumen having a substantially uniform collagen thickness, for example, about 7 minutes, thereby forming the ECM. After this patterning period, the outlet tips and inlet tips may be removed, for example, using a rotating motion to prevent rupturing of the collagen layer. The patterned channels may be filled with PBS and incubated again at about 37° C. for at least about an hour to complete the polymerization of collagen and ensure structural integrity of the formed lumen.

In some embodiments, the complex vascular architecture fabrication method comprises preparing a plurality of first cells for introduction into the parent microfluidic channel. The plurality of first cells may comprise perivascular cells such as lymphatic endothelial cells (LECs), vascular endothelial cells (VECs), lymphatic muscle cells (LMCs), vascular muscle cells (VMCs), human umbilical vein endothelial cells (HUVECs), pericytes, tissue resident immune cells, neural cells, or combinations thereof. The cells may be cultured in standard flasks confluence of at least 50%, for example, about 70% to about 80% confluence. The plurality of first cells may then be taken from the flasks using a suitable cell dissociation agent and centrifuged to collect as a pellet. The supernatant may be aspirated, and the cell pellet may then be resuspended in about 1 mL of suitable cell growth medium for cell counting. After cell counting, the cells may be centrifuged again, the supernatant may be aspirated, and the cells may be resuspended in cell growth medium at a concentration of about ten million per mL, forming a suspension comprising the first cells.

In some embodiments, prior to introducing the plurality of first cells, the parent microfluidic channel, for example, which may be previously been patterned as disclosed herein, may be filled with endothelial cell growth medium and incubated at about 37° C. in a humidified environment of about 5% CO₂ overnight, for example, to diffuse the cell media into the embedded ECM and create a favorable environment for cell attachment. A volume, for example, about 20 μL, of the suspension comprising the first cells may be injected into the patterned parent microfluidic channel, and the substrate is loaded on a rotator that rotates at about 2 rpm inside an incubator at about 37° C. and having a humidified environment of about 5% CO₂ for approximately two hours, for example, to allow an even coating of the plurality of first cells onto the lumen surface. After the two-hour incubation, the parent microfluidic channel is carefully washed with fresh cell growth medium to remove any excess cells.

In some embodiments, the seeded parent microfluidic channel may be incubated at about 37° C. in a humidified environment of about 5% CO₂ for about 24 hours under hydrostatic flow conditions to allow the plurality of first cells to become confluent within the channel and form an annular layer. The annular layer of the plurality of first cells may extend continuously and entirely circumference of the parent microfluidic channel to define a lumen extending through the parent microfluidic channel and having a complex architecture, for example, comprising a feature such as a bifurcation, tortuosity, a stenosis, or an aneurysm. The rotation of the substrate during the culturing process enables the plurality of first cells to achieve a uniform distribution around the circumference of the parent microfluidic channel, preventing the formation of asymmetrical cell layers.

In some embodiments, once the plurality of first cells has been formed into the annular layer, the parent microfluidic channel may be connected to a syringe pump to apply flow into the system for further maturation and functional assessment. The resulting annular layer of the plurality of first cells may be embedded in the ECM, for example, the layer of collagen, disposed in the parent microfluidic channel, creating living 3D vascular lumen. Additionally, in various embodiments, the annular layer of the plurality of first cells defines a lumen having a complex architecture, for example, comprising a feature such as a bifurcation, tortuosity, a stenosis, or an aneurysm.

In some embodiments, the complex vascular architecture fabrication method comprises preparing a plurality of second cells that are different from the plurality of first cells for integration into the parent microfluidic channel during the gravitational lumen patterning process. The plurality of second cells may comprise lymphatic muscle cells (LMCs), vascular muscle cells (VMCs), or combinations thereof. The plurality of second cells may be cultured in standard flasks until achieving suitable confluence, then taken from flasks using a suitable cell dissociation agent and centrifuged to collect as a pellet. The supernatant may aspirated, and the cell pellet may be resuspended in a suitable cell growth medium for cell counting. After cell counting, the cells may be centrifuged again, the supernatant may be aspirated, and the cells may be resuspended at an appropriate concentration for mixing with the collagen solution.

In some embodiments, the method may further comprise culturing the plurality of the second cells that are different from the plurality of first cells in the parent microfluidic channel such that an annular layer of the plurality of second cells is formed in the parent microfluidic channel. Unlike the plurality of first cells, the plurality of second cells may be mixed with the collagen solution prior to injection into the parent microfluidic channel, enabling the formation of an annular outer layer of muscle cells embedded within the extracellular matrix during the subsequent gravitational lumen patterning process. This integration of the plurality of second cells with the collagen matrix may occur before positioning the substrate in the vertical orientation and before the introduction of the plurality of first cells. For example, the layer of the plurality of first cells may form an annular inner layer in the parent microfluidic channel and the layer of the plurality of second cells may form an annular outer layer in the parent microfluidic channel that is radially positioned between the inner layer and the inner surface of the parent microfluidic channel.

In some embodiments, the plurality of first cells and the plurality of second cells may have specific alignments relative to the axial direction of extension of the parent microfluidic channel. For example, when the plurality of first cells comprise endothelial cells, a majority of the endothelial cells may align substantially parallel to the longitudinal direction of extension of the parent microfluidic channel, while when the plurality of second cells comprise muscle cells, a majority of the muscle cells may align substantially perpendicular to the longitudinal direction of extension of the parent microfluidic channel. This orthogonal alignment pattern mimics the physiological orientation observed in vivo vessels, where endothelial cells align with flow direction and muscle cells wrap circumferentially around the vessel.

In some embodiments, the lumen defined by the annular layer of the plurality of first cells may be characterized as having specific geometric characteristics that enhance physiological relevance. For example, the lumen may have an elliptical cross-section defined by a major axis and a minor axis extending orthogonal to the major axis, wherein each axis intersects the parent microfluidic channel perpendicular to a direction of axial extension of the parent microfluidic channel at the point of intersection. A ratio of the major axis to the minor axis may be between about 1.1:1 and about 5:1, between about 1.1:1 and about 3:1, or between about 1.5:1 and about 2.5:1. Additionally, the lumen may range approximately from about 10 microns to about 800 microns in diameter.

In some embodiments, the complex vascular architecture fabrication method may further comprise applying fluid flow through the formed lumen to promote cellular maturation and assess vessel functionality. For example, a reservoir containing fresh cell growth medium may be connected to the inlet of the parent microfluidic channel, and laminar flow may be applied at a rate of about 1 μL per minute for approximately 24 hours to promote proper cell alignment and maturation. This flow application may enable assessment of the hemodynamic properties of the complex vascular architectures and validation of their physiological relevance.

In some embodiments, the complex vascular architecture fabrication method comprises validating and characterizing the formed vessels to demonstrate the functionality of the complex vascular architectures through various analytical techniques. For example, the validation process may include fluorescent staining methods to assess cellular organization and barrier function. Endothelial cells may be fixed with about 4% paraformaldehyde for about 15 minutes at room temperature to preserve cellular components and physical characteristics. The cells may then be permeabilized with about 0.1% Triton X-100 in blocking buffer for about 30 minutes at room temperature to allow staining molecules to enter the cells. For cellular structure visualization, rhodamine phalloidin may be mixed with blocking buffer in a ratio of about 1:100 to stain filamentous actin proteins within the cells, and VE-cadherin primary antibody may be diluted in blocking buffer at about a 1:200 ratio to assess cell-cell junctions and barrier integrity. Additionally, Hoechst may be mixed with PBS in about a 1:2000 ratio to label cell nuclei. The validation process may further comprise perfusion testing using diluted red blood cell suspensions to assess vessel patency and flow characteristics. Whole human blood may be mixed with equal parts PBS, centrifuged at about 400 g for about 10 minutes, and the process repeated to remove plasma clotting factors, with the pelleted RBCs finally diluted to achieve a hematocrit content of about 2-4% prior to perfusion through the vessels at about 40 μL per minute. The vessels may also be characterized through computational fluid dynamics analysis to assess hemodynamic properties, including time averaged wall shear stress (TAWSS) and relative residence time (RRT), using physiological flow conditions with cardiac pressure waveforms to validate the pathological hemodynamics that are witnessed in vivo.

In some embodiments, the complex vascular architectures fabricated according to the disclosed methods may be utilized in a variety of applications where the physiologically relevant structural and functional characteristics may be particularly advantageous. For example, the disclosed in vitro vessels with complex architecture, which may be referred to as a vessel-on-chip, may serve as a platform for drug screening applications, particularly for investigating vascular complications associated with vessel structure such as aneurysm, fibromuscular dysplasia, and atherosclerosis. The complex architectures enable more accurate assessment of drug transport and human in vivo vasculature responses compared to simple cylindrical vessel models, for instance, because the spatially heterogeneous shear stress distribution and altered flow dynamics more accurately model endothelial cell responses. For disease modeling applications, the disclosed vessel-on-chips may be used to investigate vascular complications like aortic and cerebral aneurysm, arterial tortuosity syndrome, atherosclerosis, and carotid artery disease, where vessel architecture plays a crucial role in disease onset and progression. Additionally, the ability to create in vitro vascular architectures specifically modeled to a particular patient enables personalized medicine applications, including personalized therapeutic drug testing and development. Additionally, the complex vessel-on-chips may be utilized to examine inflammatory behavior and associated immune responses at diseased sites, including leukocyte rolling and transmigration in post-stenosed regions of atherosclerosis, and to investigate developmental processes like lymphatic valve formation in response to flow oscillations. The disclosed complex vessel-on-chips may also enable development of therapeutic strategies that not only target molecular and cellular processes that occur in vivo, but also can seek to limit or reverse pathological flow behavior through evaluation of alternate, less invasive treatment strategies that focus on flow correction or rehabilitation.

In some embodiments, the complex vascular architectures fabricated according to the disclosed methods may be integrated into systems for modeling vascular disease and conducting drug screening applications. Such systems may comprise the in vitro vessel having complex architecture as disclosed herein, combined with fluid perfusion systems configured to establish controlled flow through the lumen, and imaging systems configured to monitor cellular responses within the complex vascular architecture. The fluid perfusion systems may be configured to apply a TAWSS value ranging from 0.1 to 10 dynes/cm² to simulate physiological and pathological flow conditions. In various embodiments, perfusion may include perfusion of blood and blood components, including platelets, immune cells, red blood cells or their combinations. Additionally or alternatively, in some embodiments, perfusion may also include perfusion of drugs, molecules, toxins, chemicals, and other particulates relevant to transport in blood or lymphatic vessels in vivo or used in preclinical research. Additionally, these systems may further comprise computational fluid dynamics modules configured to predict hemodynamic parameters within the complex vascular architecture, enabling real-time analysis of flow patterns, shear stress distribution, and particle residence times. The integration of these components creates a platform for investigating vascular complications, drug transport mechanisms, and endothelial cell responses under controlled conditions that closely mimic in vivo environments. Such systems may enable researchers to systematically study the effects of complex vessel geometries on cellular behavior, drug efficacy, and disease progression in a controlled laboratory setting.

### EXAMPLES

The following are examples related to a vessel-on-chip having complex vascular architecture as disclosed herein.

### Materials and Methods

Complex vessel designs and 3D mold production. Complex microfluidic channels, inspired by clinically observed vascular malformations, were designed to model bifurcations, tortuosity, stenosis, and aneurysms. Each pathological vessel was designed with multiple variations by adjusting angles, tortuosity levels, degrees of stenosis, or extents of expansion, respectively, using computer aided design (CAD) in Autodesk Fusion360. Briefly, bifurcation channels were designed using 500 μm×500 μm channels that branched into two 350 μm×500 μm channels, with 30°, 60°, and 90° bifurcation angle variations. Tortuous channels were designed using 500 μm×500 μm straight channels with peak-to-peak heights of 1.5 mm, 2 mm, and 3 mm. Stenosis models were designed using 500 μm×500 μm cross-section channels, containing three equally spaced constrictions of 25%, 50%, and 75% widths. Similarly, aneurysm channels were designed using 500 μm×500 μm channels containing 200% or 400% expansion of the cross-sectional widths. All channels were designed to have an end-to-end length of 2 cm. CAD designs for all vessel variations were exported as stereolithography (STL) files and were finally printed using an Asiga Max SLA printer and clear resin, particularly, audioprint® GR-10. After printing, molds were sonicated in 100% isopropyl alcohol (IPA) for 30 minutes to remove excess uncured resin, followed by UV treatment in an inert, nitrogen gas (N₂) environment to complete resin curing.

Microfluidic device fabrication. Microfluidic channels were patterned using the designed molds via soft lithography using polydimethylsiloxane (PDMS). Briefly, PDMS prepolymers were mixed with the curing agent in a 10:1 ratio. The prepared PDMS mixtures were poured into the molds, followed by degassing in a vacuum desiccator to remove traces of air. PDMS containing molds were then placed in a 70° C. oven for at least two hours to complete curing. Once the polymerization process was completed, the PDMS stamps were carefully peeled off from the molds. Inlet and outlet ports were created at the ends of each channel using a 1 mm biopsy punch from Miltex-Integra. Excess PDMS was trimmed and the PDMS stamps were bonded to PDMS coated glass slides using 75 watts oxygen plasma at 0.80 mbar for 30 seconds. Once bonded, devices were stored in a 70° C. oven overnight under weighted conditions to complete the bonding process, resulting in irreversibly bonded, completely sealed channels.

Chemical pretreatment. Fabricated microfluidic channels were chemically pretreated to enhance bonding of the extracellular matrix (ECM) and PDMS. Briefly, microfluidic channels were first oxygen plasma-treated to activate the PDMS surfaces. Once treated, the devices were immediately injected with a 10% v/v solution of 3-aminopropyltriethoxysilane (APTES) in 200 proof ethanol. Channels were incubated for 15 minutes at room temperature before thorough rinsing with 100% and 70% ethanol solutions, respectively. Channels were completely dried with air to remove any traces of fluid and channels were then incubated in a 70° C. oven for 30 minutes. Channels were then injected with a 2.5% v/v solution of glutaraldehyde in PBS, followed by incubation at room temperature for 30 minutes. Channels were then extensively washed with 100% and 70% ethanol solutions to remove any traces of leftover glutaraldehyde. Channels were allowed to dry overnight in an oven and then degassed in a vacuum desiccator for at least two hours prior to collagen patterning.

Gravitational lumen patterning. Collagen solutions were prepared according to the manufacturer's protocol. The collagen stock (rat tail type 1; 8.50 mg mL⁻¹, Corning; 10 mg mL⁻¹, Ibidi) was reconstituted to a concentration range of 5 to 7.5 mg mL⁻¹, with the pH adjusted to 7.5 using 7.5% sodium bicarbonate from Gibco, sodium hydroxide from Thermo Fisher, and 1 M HEPES from Gibco. For lumen visualization studies, the PBS solution was mixed with red fluorescent microbeads and was used in place of plain 1× PBS. Collagen mixtures were prepared on ice to prevent premature cross-linking of collagen solutions. GLP was then pursued as per previously reported methods. Briefly, prior to injecting collagen solutions into microfluidic channels, 200 μL pipette tips (P200) were bent to nearly 135° roughly 7 mm into the tip length. Individual bent tips were pushed into the outlet ports of the channel till tips reached the top of the microfluidic channels, except for bifurcated channels, where two curved tips were employed due to the binary splitting. Using a 20 μL pipette, ice-cold collagen was injected into the microfluidic channels until the levels of collagen were roughly equal between the inlet and outlet. Collagen was allowed to equilibrate for 15 seconds after which inlet tips were carefully removed. Simultaneously, another bent P200 tip was loosely connected to a 200 μL pipette and 40 μL of 1× PBS was aspirated. Devices were then held vertically with the outlet ports at the bottom, and PBS containing bent tips were connected to the inlet. 40 μL of PBS was used for fabricating all vessels, except for straight channels where 10 μL, 20 μL, and 40 μL of PBS were used for fabricating vessels with varying diameters. Once tips with PBS were pushed in the respective inlets, devices were immediately moved to a 5% carbon dioxide (CO₂) 37° C. humidified incubator while holding devices vertically. Devices were incubated for 7 minutes, after which devices were brought back to biosafety cabinets. The outlet tips were gently removed first followed by inlet tips. Tips were removed using a rotating motion to prevent the rupturing of the collagen layer. Patterned channels were filled with PBS and incubated again in the 37° C. incubator for at least an hour to complete the polymerization of collagen.

Preparation of the diluted RBC suspension. Whole human blood was mixed with equal parts 1× PBS and centrifuged at 400 g for 10 minutes. The supernatant was discarded, and the process was repeated twice to remove any trace of residual plasma, the presence of which would initiate clotting due to the interaction with collagen within the GLP lumen. Finally, the pelleted red blood cells (RBCs) were diluted to achieve a hematocrit content of 2-4% prior to addition to the vessels and perfused through the GLP vessels at 40 μL min-1 using a syringe pump from Harvard Apparatus.

Endothelial cell culture. Patterned vessels were filled with endothelial cell growth medium 2 (EGM-2) from PromoCell and incubated at 37° C. in a humidified 5% CO₂ incubator overnight to diffuse the cell media into the embedded ECM and create a favorable environment for endothelial cell attachment. HUVECs from PromoCell were cultured in standard T75 flasks until 75-80% confluence. HUVECs were then lifted from flasks using Accutase from Thermo Fisher and centrifuged to collect as a pellet. The supernatant was aspirated, and the cell pellet was resuspended in 1 mL EGM-2 for cell counting. After cell counting, cells were centrifuged again, and supernatant was aspirated. The cells were finally resuspended in EGM-2 at a concentration of 10 million per mL. 20 μL of the prepared cell suspension was injected into the patterned vessels, and devices were loaded on a rotator that rotates at 2 rpm inside a 37° C. humidified 5% CO₂ incubator for approximately two hours to allow an even coating of endothelial cells to the lumen surface. After the two-hour incubation, vessels were carefully washed with fresh EGM-2 to remove excess cells. The seeded vessels were incubated at 37° C. in a humidified 5% CO₂ incubator for 24 hours under hydrostatic flow conditions to allow the cells to become confluent within the channel.

Cell staining. After the shear exposure, endothelial cells were fixed with 4% paraformaldehyde (PFA) for 15 minutes at room temperature. Vessels were then permeabilized with a 0.1% solution of Triton X-100 in blocking buffer (2% bovine serum albumin (BSA)/Dulbecco's phosphate-buffered saline (DPBS)) for 30 minutes at room temperature. For VE-cadherin staining, mouse anti-human VE-cadherin primary antibody from R&D Systems was diluted in blocking buffer at a 1:200 ratio. 20 μL of the primary antibody solutions were injected into each vessel and incubated at 4° C. overnight. After incubation, the primary antibody solutions were washed with blocking buffer solution and then a 1:200 solution of secondary rabbit anti-mouse antibody from R&D Systems in blocking buffer was incubated within the vessels for 2 hours at room temperature. Vessels were then washed with blocking buffer thoroughly. To stain nuclei, Hoechst 33342 from Thermo Fisher was mixed with PBS in a 1:2000 ratio. 20 μL of the staining solution was injected into the vessel and incubated for 30 minutes at room temperature before rinsing with PBS. The stained vessels were filled with fresh PBS and stored at 4° C. overnight.

Orientation and morphological analysis. Once fluorescence staining images of VE-cadherin structures were obtained, the boundaries of 20 cells in each region of interest were manually outlined using ImageJ, and the cell area and perimeter of each cell were measured. The cell shape index (CSI) was calculated using eqn (1). Angles were computed for the major axis of cells with respect to flow directions and plotted as polar plots of angular distribution using a python code.

3D CAD reconstruction and CFD analysis. 3D z-stack images of the vessels were imported in Autodesk Fusion 360 and 3D representations of each vessel with all variations were produced. The 3D files were exported in the STEP file format which were then imported into COMSOL Multiphysics®. Once imported, the laminar flow module was used to model the hemodynamic flow, and a cardiac cycle waveform was used as the inlet pressure boundary condition. The period of the waveform was 1 second and a Fourier series approximation for the cardiac cycle was used. In the computational model, blood was simulated as a Newtonian fluid with a viscosity of 3.5 cP, and a no-slip boundary condition was used along all exterior walls. A temporal simulation was then performed after discretizing and meshing the 3D vessel domains and ancillary variables like time averaged wall shear stress (TAWSS) and relative residence time (RRT) were calculated. TAWSS and RRT were calculated for the entire length of vessels, and not just in the target regions such as stenosis or aneurysm. The calculated TAWSS was normalized with respect to the TAWSS values obtained by applying the same boundary conditions across a simple, cylindrical lumen with the same inlet diameter and end-to-end length as that of the respective vessel variation analyzed. The calculated RRT was normalized in the same manner as TAWSS.

Statistical analysis. All data were reported as mean±standard error of mean (SEM). All experiments were performed in triplicate (n=3) unless otherwise stated. Statistical comparisons were made using two-way ANOVA, repeated measures two-way ANOVA, Student's t-test or Pearson correlation test in GraphPad Prism ver. 9. Differences were considered significant for p<0.05. Multiple testing correction was performed using the Tukey test in GraphPad Prism ver. 10.

### Results and Discussion

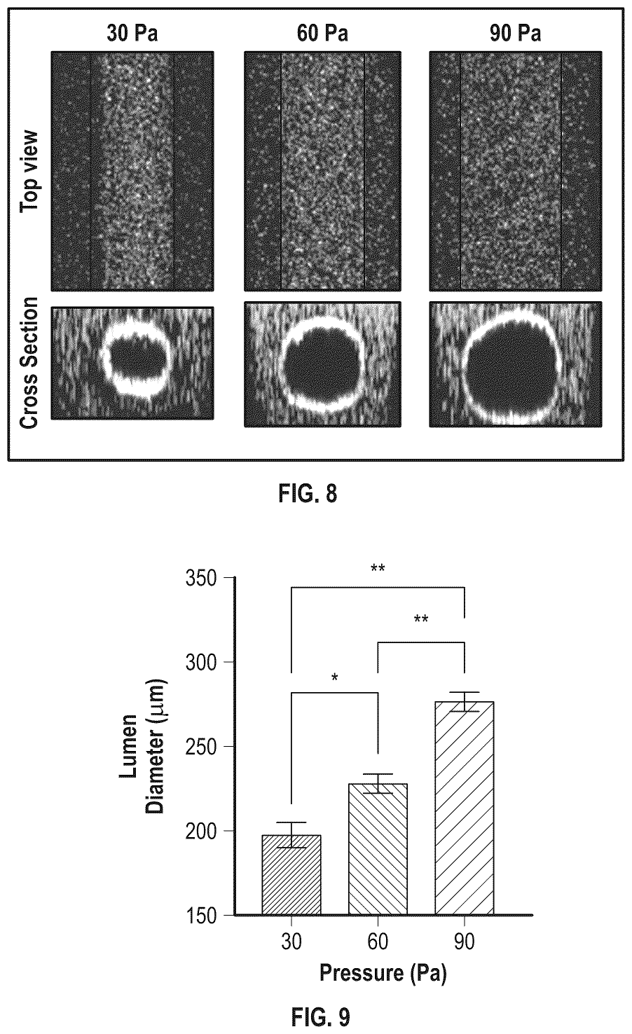

Engineering vascular architecture-on-a-chip: cylinders, aneurysm, stenosis, bifurcations and tortuosity. Simple, uniform vessels were first engineered by modulating the pressure head during the GLP process. When GLP was performed in straight microfluidic channels filled with reconstituted collagen and layered different amounts of PBS, it was observed that there is an increase in vessel diameter as the pressure head increases, as shown in FIGS. 8 and 9. Using pressure heads that can be applied easily in these devices, vessel-chips of diameters in the range of 150-300 μm were able to be formed. As shown in FIG. 2B, lumen diameters increased from approximately 200 μm at 30 Pa to 280 μm at 90 Pa, with statistical analysis revealing significant differences between diameter groups (p<0.005).

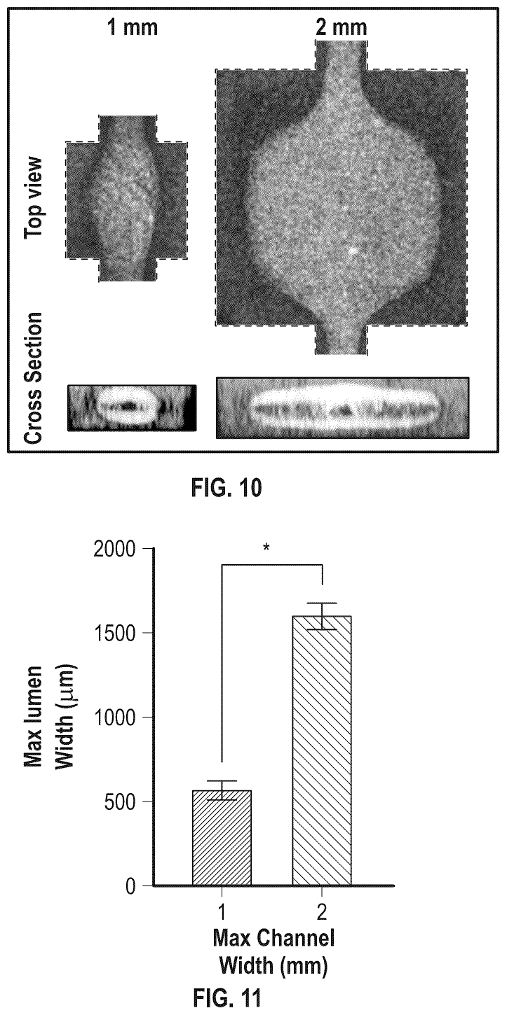

Microfluidic channels with 1 mm (200%) and 2 mm (400%) expansions were constructed and patterning was executed to create aneurysms-on-chip, as shown in FIG. 10. The extents of aneurysms followed the trend of their pre-GLP microchannels as each variation yielded aneurysms with nearly ˜200% and 400% of the pre-aneurysm diameters, as shown in FIG. 11. Maximum lumen widths reached approximately 1500 μm for the 1 mm expansion and 2000 μm for the 2 mm expansion channels, with statistically significant differences observed (p<0.0001) between the 1 mm and 2 mm expansion conditions as shown in FIG. 2D.

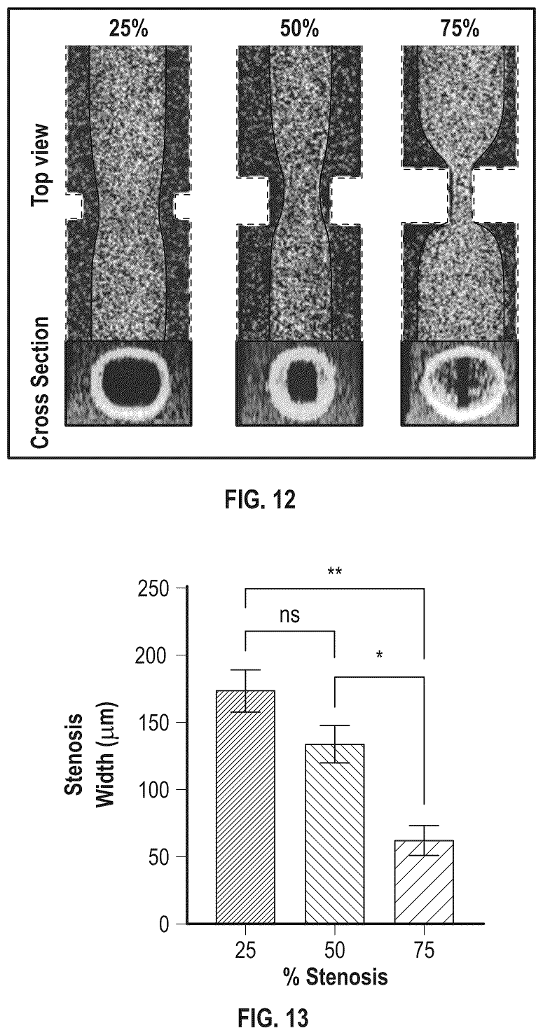

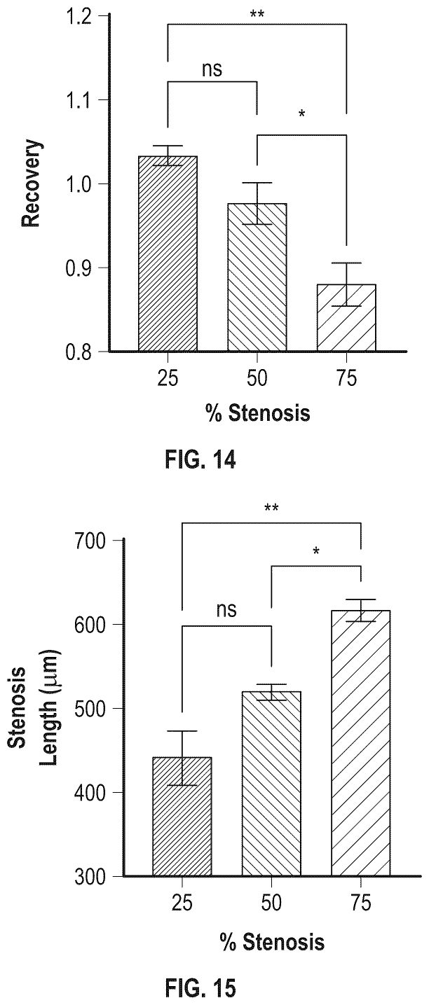

For stenosis-on-chip fabrication, differential extents of stenosis using 25, 50 and 75% constriction channels were observed, as shown in FIG. 12 and FIG. 13. Quantification revealed stenosis widths ranging from approximately 50 μm for 75% stenosis to 250 μm for 25% stenosis, with significant differences between groups (p<0.005; p<0.05) as demonstrated in FIG. 13. Lumen recovery ratios were distributed around unity (perfect recovery) ranging from 0.8 to 1.1, with no significant differences (ns) between most groups, as shown in FIG. 14. Stenosis lengths ranged from approximately 300 μm to 700 μm, showing significant differences between the 25% and 75% stenosis groups (p<0.005; p<0.05) as shown in FIG. 15.

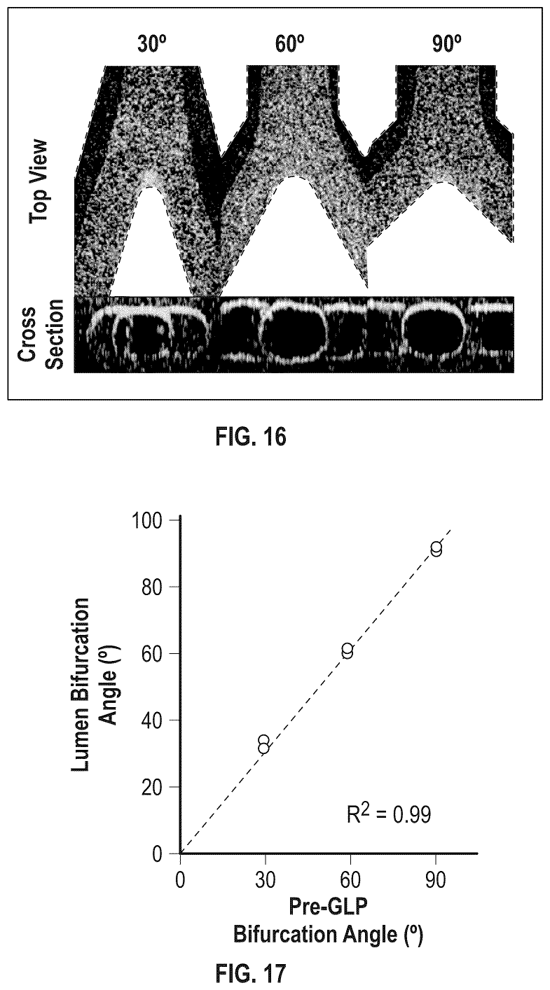

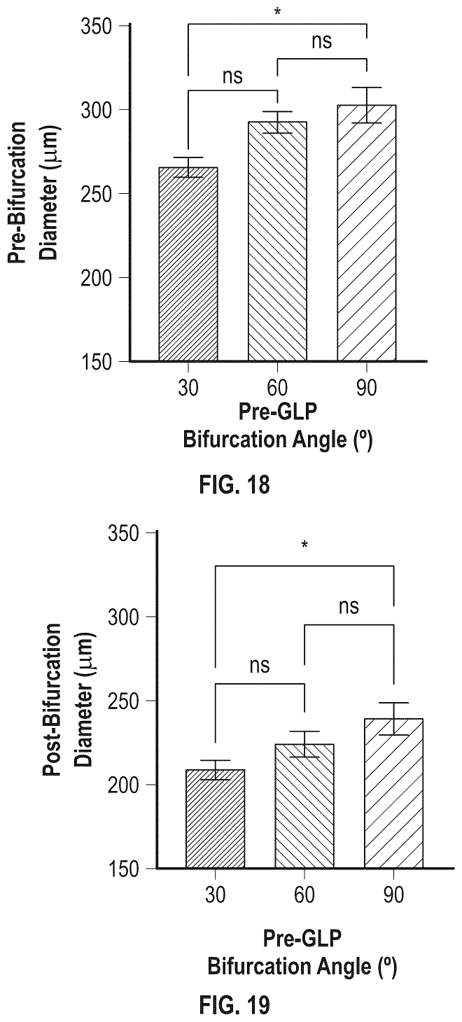

When bifurcation-on-chip structures were created, as shown in FIG. 16, the bifurcation angles of the lumen were quantified and regression analysis was performed, achieving high correlation (R2=0.99) between pre-GLP and post-GLP angles as shown in FIG. 17. Pre-bifurcation diameters ranged from approximately 200 μm to 350 μm, with significant differences observed (p<0.05; ns: not significant for some comparisons) as demonstrated in FIG. 18. Post-bifurcation diameters were reduced to approximately 150 μm to 300 μm, as shown in FIG. 19. The reduction in post-GLP lumen diameter was typically 23%, while the reduction in the width of the pre-GLP microchannel was 30%.

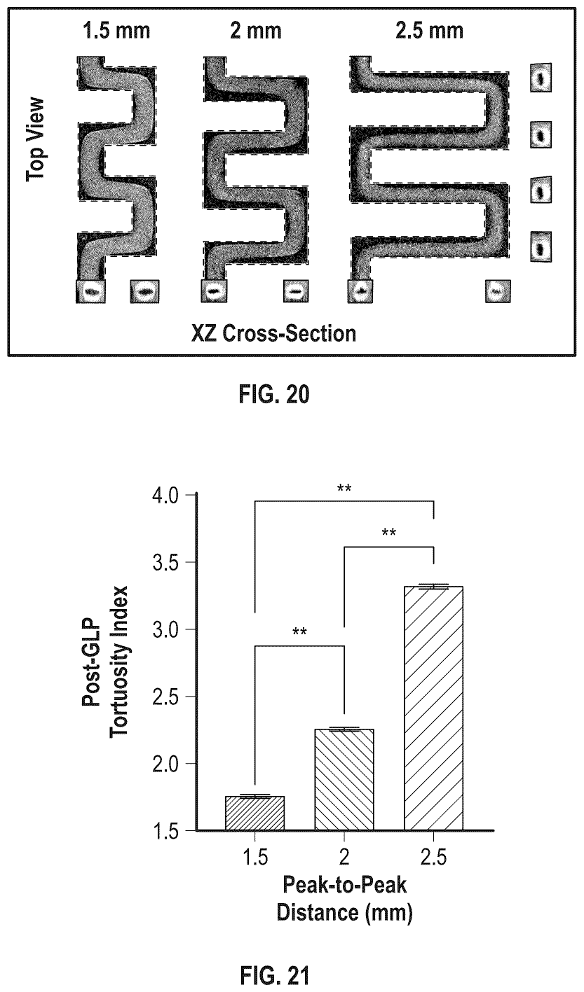

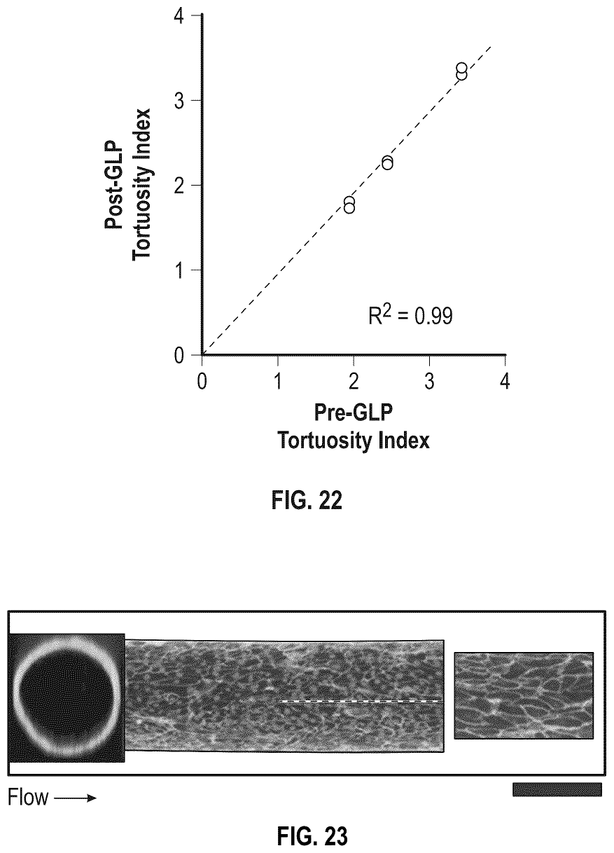

For tortuous vessel-chips, as shown in FIG. 20, regression analysis on the tortuosity index before and after GLP achieved high correlation (R2=0.99) as shown in FIG. 22. Tortuosity indices ranged from approximately 1.5 to 4.0, with significant differences between groups (p<0.005; p<0.0001) as demonstrated in FIG. 21. The normalized tortuosity ratios were distributed around unity, with some variations showing significant differences (p<0.05; p<0.005; ns: not significant).

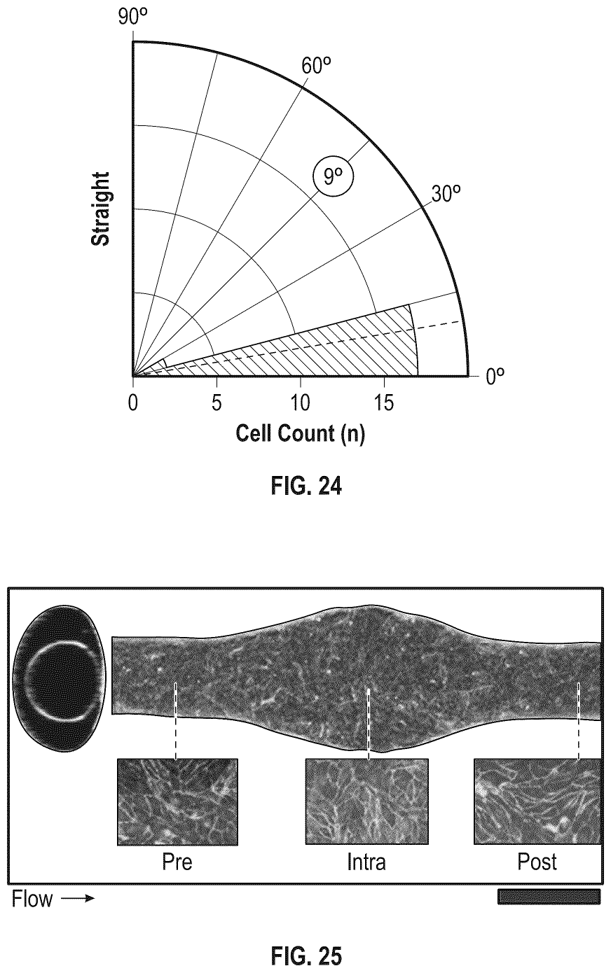

Endothelialization and orientation analysis of the complex vessel-chips. For cylindrical vessel-chips, endothelial cells created a confluent vascular lumen covered with cells on all sides, as shown in FIG. 23. The cells displayed alignment along the axial flow directions with mean cell counts distributed around 0° as shown in the polar plot, as shown in FIG. 24.

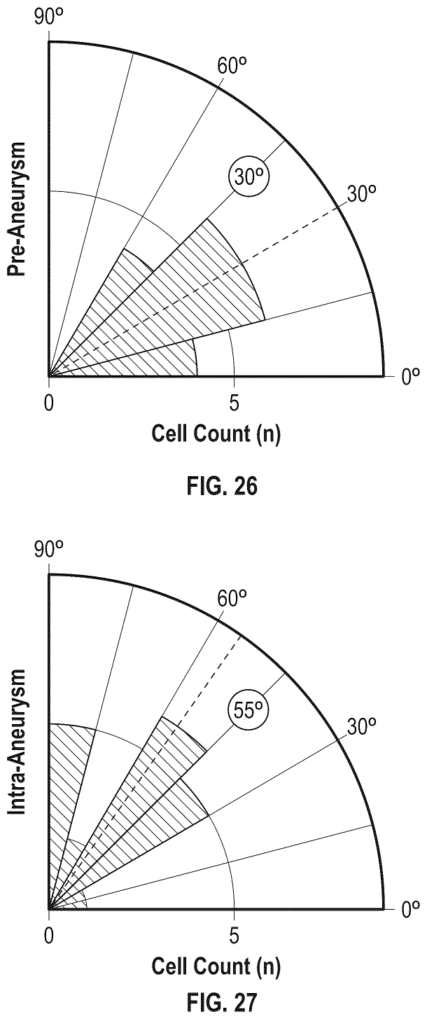

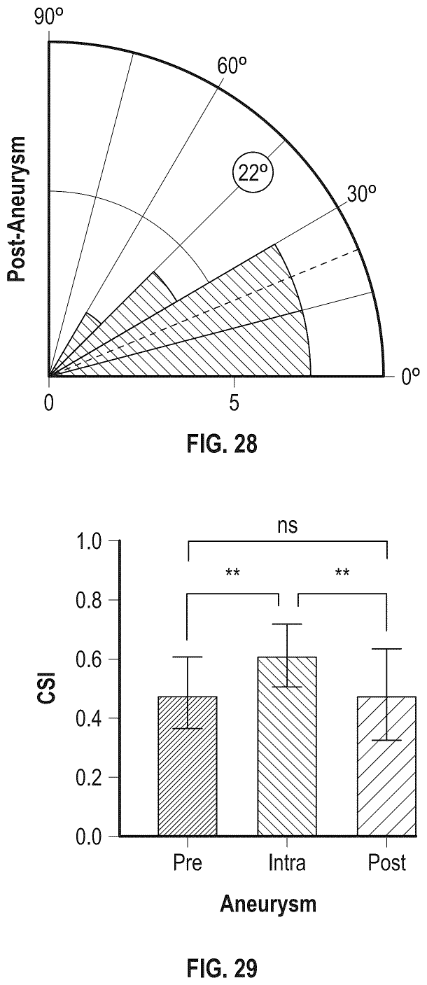

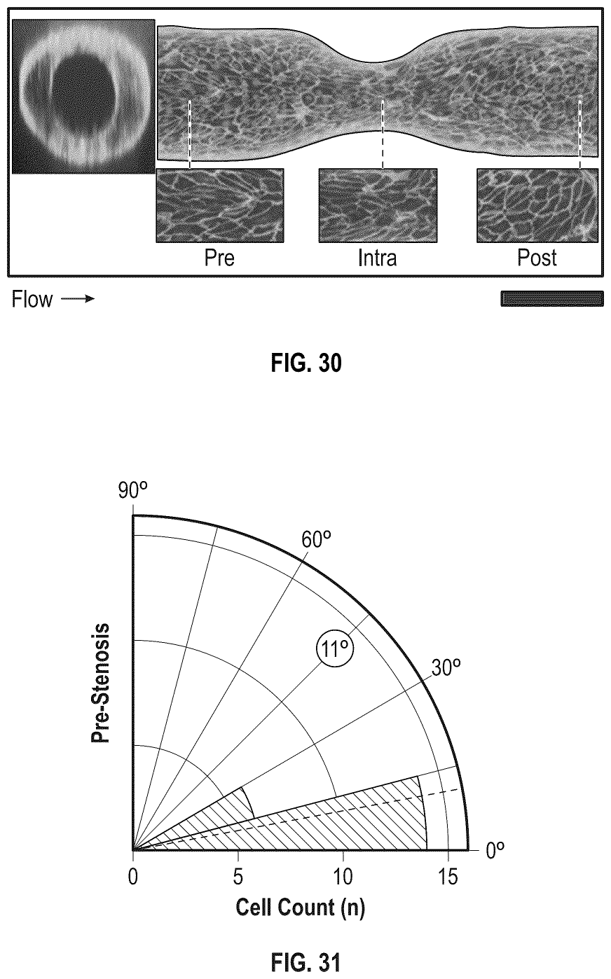

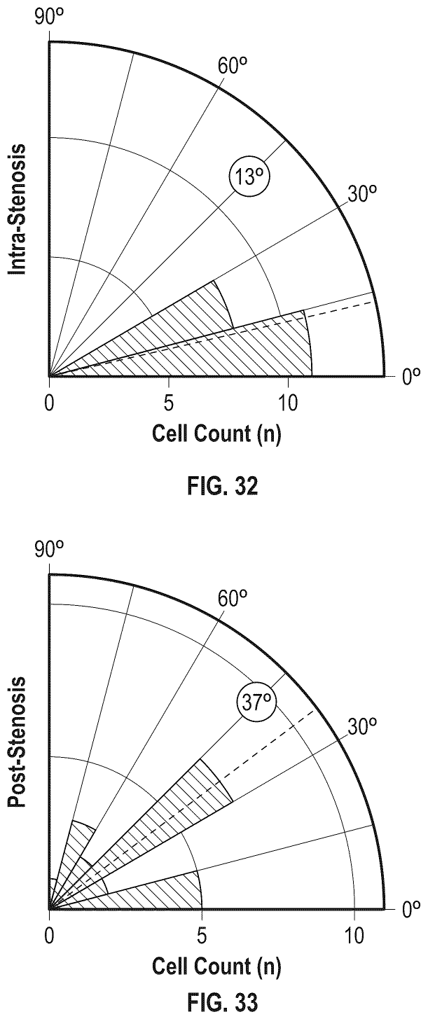

For aneurysms-on-chip, cell shape index (CSI) analysis revealed that cells at the aneurysm exhibit a more rounded shape (CSI approximately 0.4-0.6) compared to the pre- and post-aneurysm sections (CSI approximately 0.8-1.0), with significant differences observed (p<0.005) as shown in FIG. 29. Cell orientation analysis showed mean angles of 55° in pre-aneurysm regions, 22° at aneurysm sites, and 30° in post-aneurysm regions as demonstrated in FIG. 26 and FIG. 28.

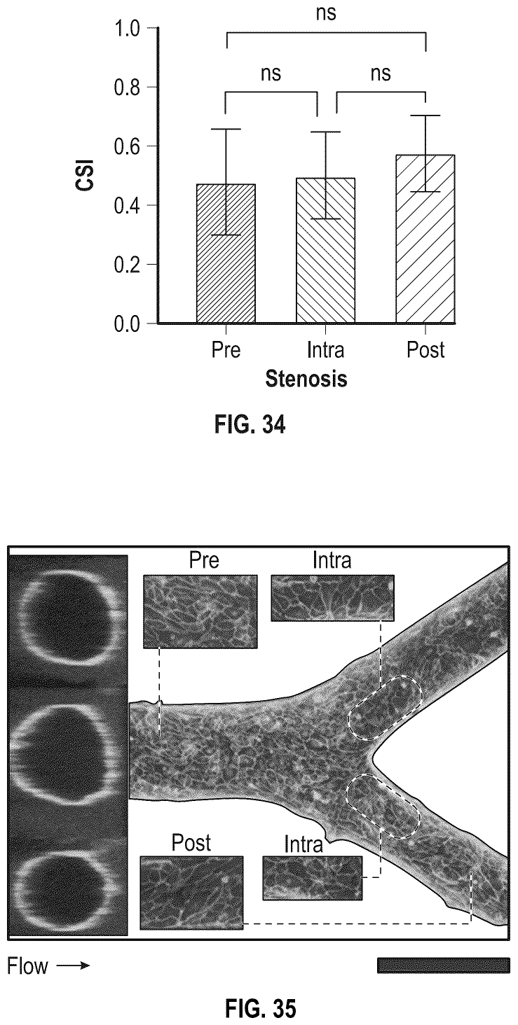

In stenosis-on-chip structures, cell orientation analysis showed mean angles of approximately 11° in pre-stenosis regions, 13° in stenosis regions, and 37° in post-stenosis regions as shown in FIG. 31 and FIG. 33. CSI values remained consistent across all regions (approximately 0.6-1.0) with no significant differences (ns) observed between regions as demonstrated in FIG. 34.

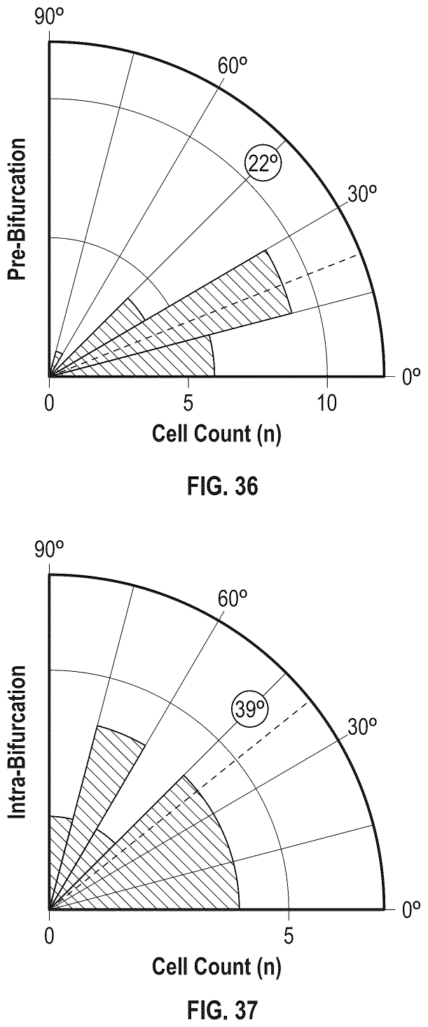

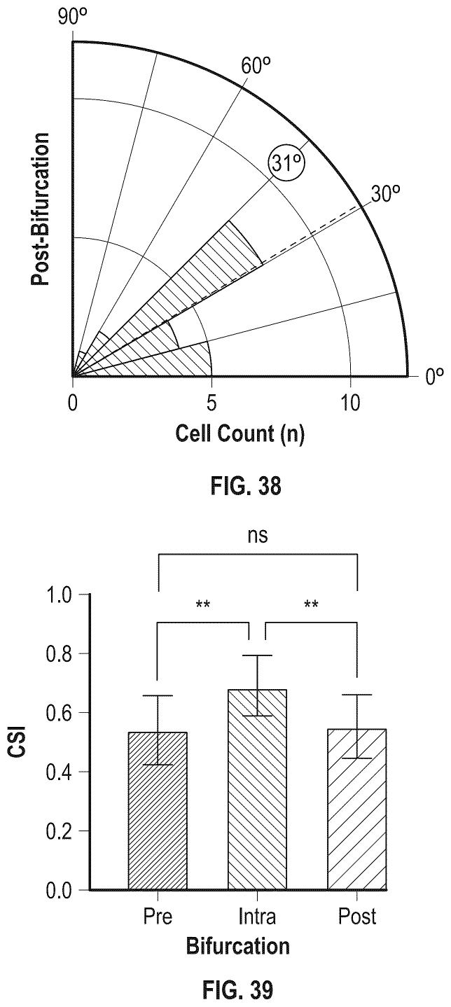

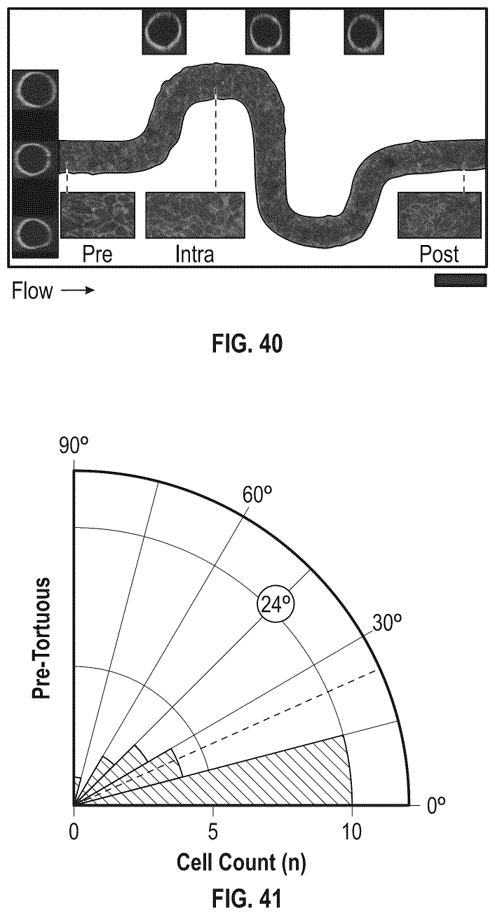

For bifurcation-on-chip analysis, cell orientation analysis revealed mean angles of 22° in pre-bifurcation regions, 39° at bifurcation sites, and 31° in post-bifurcation regions as shown in FIG. 36, FIG. 37 and FIG. 38. CSI analysis showed significant differences (p<0.005) between pre-bifurcation (CSI ˜0.8-1.0) and bifurcation regions (CSI ˜0.4-0.6), with cells at bifurcation sites displaying more rounded morphology as demonstrated in FIG. 39.

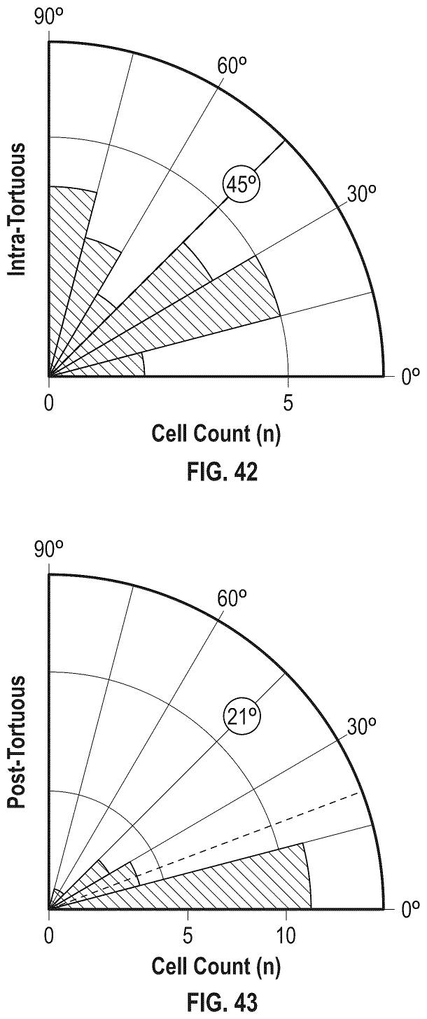

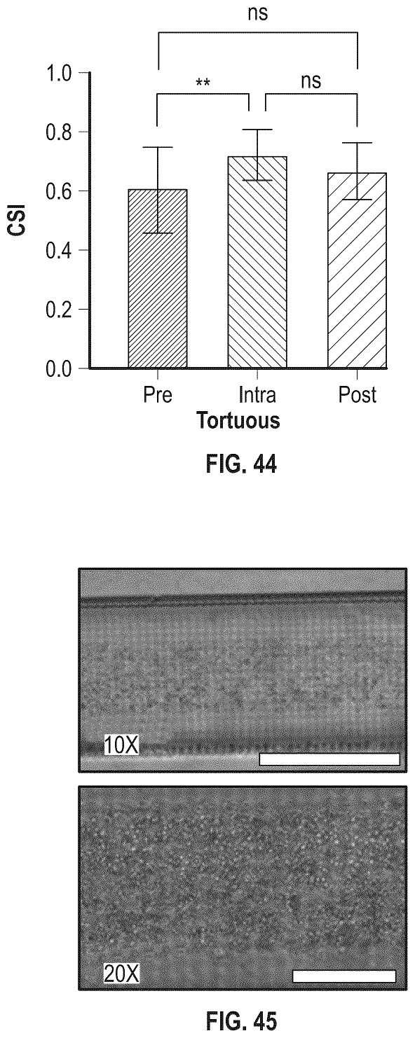

In tortuous vessel-chips, cell orientation analysis showed mean angles of 24° in pre-tortuous regions, 45° in tortuous sections, and 21° in post-tortuous regions as shown in FIGS. 41, 42, and 43. CSI analysis revealed significant differences (p<0.05) between pre-tortuous (CSI ˜0.8-1.0) and tortuous regions (CSI ˜0.6-0.8) as demonstrated in FIG. 44.

Hemodynamic analysis of the complex vessels. Complex vascular architecture also results in spatially heterogeneous shear stress distribution among these structures that may result in unique endothelial responses to flow. Therefore, blood was also introduced in these structures and a shear stress distribution atlas was prepared using computational predictions useful for future structure-function studies.

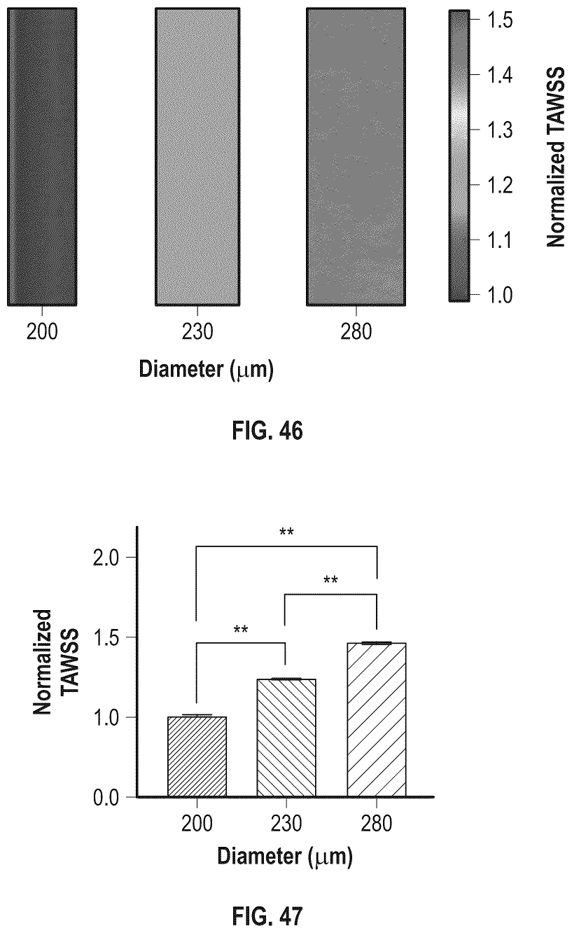

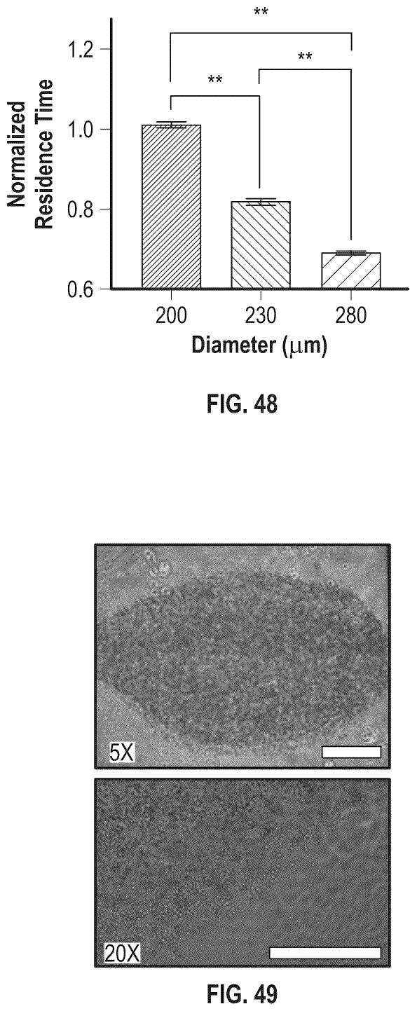

For cylindrical vessel-chips, the vessels were first exposed under the perfusion of red blood cells (RBCs) for approximately half an hour, and no loss in lumen integrity was observed, as shown in FIG. 45. Since microphysiological flows are mostly laminar, well-established computational algorithms could be applied to predict hemodynamics in these vessels and validate flow patterns. As expected for a uniform flow in a cylinder, the computations predicted a uniformly distributed time-averaged wall shear stress (TAWSS) for all diameter variations of the vessel-chips, as shown in FIG. 46. Normalized TAWSS values increased linearly from approximately 1.0 to 1.5 as vessel diameter increased from 200 μm to 280 μm, with significant differences observed (p<0.005) as shown in FIG. 47. Normalized residence time decreased correspondingly from approximately 1.2 to 0.6 as diameter increased, as shown in FIG. 48.

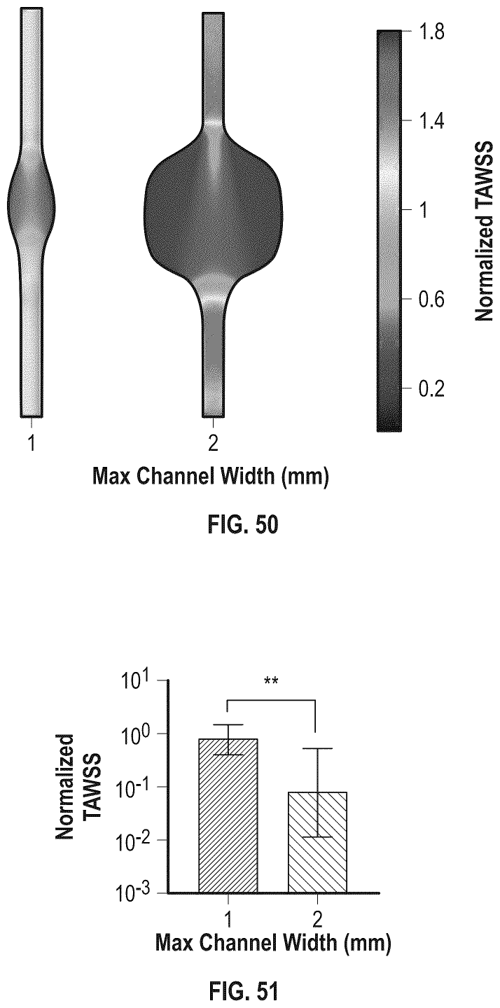

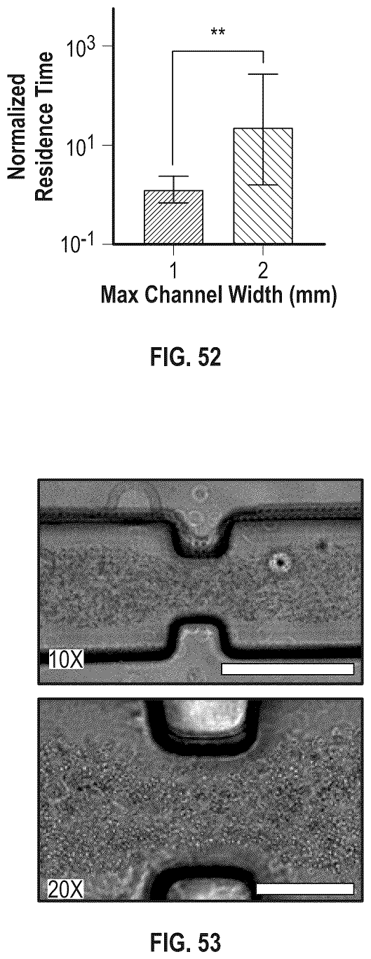

For aneurysms-on-chip, aneurysms are typically characterized by stagnation and reduced flow at the extremities. To test if the adhered ECM within the engineered structures supports the flow of RBCs and validate the reduced hemodynamics at the extreme sections in the aneurysm-chip, the aneurysm-chips were exposed under perfusion and it was observed that RBCs in the extremes of the aneurysms moved slower in comparison to those in the middle, as shown in FIG. 49. This experimental observation was supported by the computational analysis which also revealed a significantly reduced flow in the extremes of aneurysms, as shown in FIG. 50. This was consistent with the in vivo findings where extremely low TAWSS values have been reported along with fluid recirculation. TAWSS analysis revealed values ranging from 10⁻¹ to 10¹ (normalized), with significant differences between 1 mm and 2 mm expansion conditions (p<0.005) as shown in FIG. 51. Normalized residence time values ranged from 10¹ to 10⁵, showing significant differences between expansion conditions (p<0.005) as demonstrated in FIG. 52.

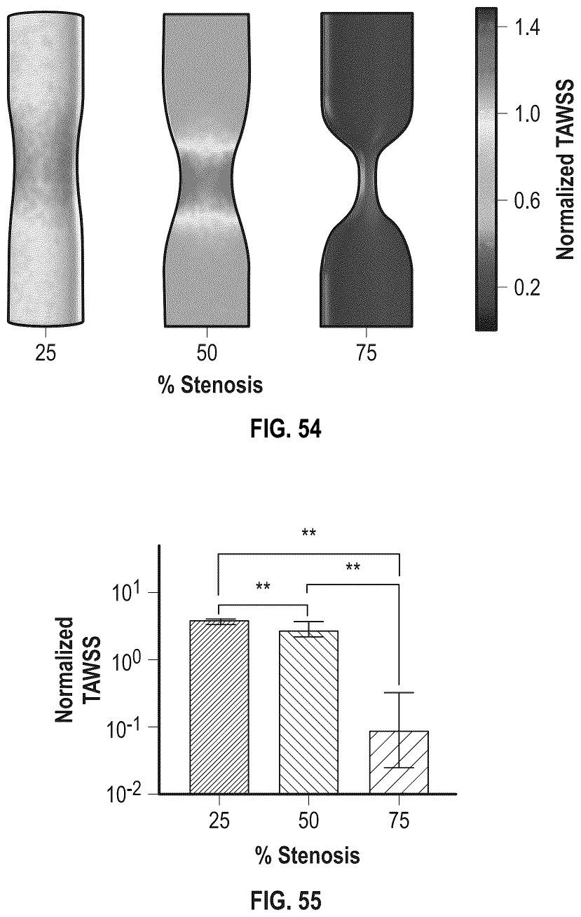

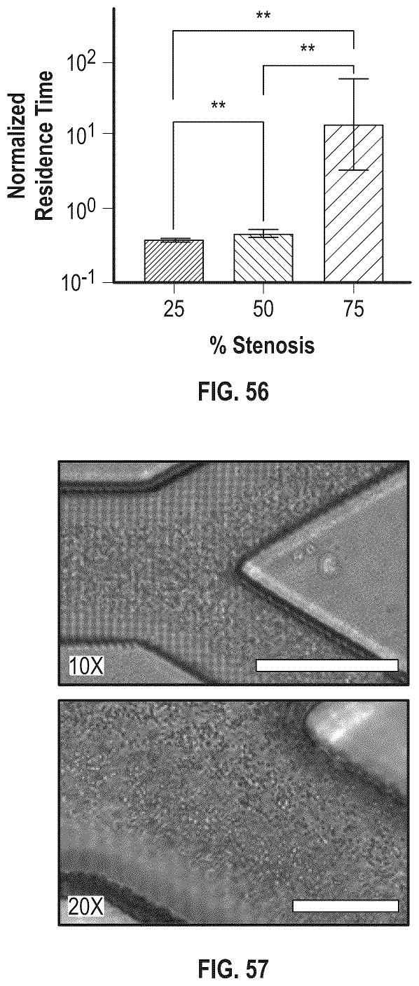

For stenosis-on-chip analysis, RBCs were next introduced within the stenosed vessels and these stenosed sections supported perfusion for half an hour without any change in dimensions, as shown in FIG. 53. To validate the influence of stenosed geometry on flow, fluid dynamics simulations were also performed under physiological flow conditions assuming a cardiac pressure waveform and the TAWSS was calculated, as shown in FIG. 54 and it was observed that increasing extents of stenosis had increasing TAWSS values in the stenosis regions. The TAWSS values decreased in the pre- and post-stenosis regions as the extent of stenosis increased due to more resistance to fluid flow and hence lower flow rates in these regions. TAWSS values ranged from 10⁻² to 10² (normalized) across different stenosis percentages, with significant differences observed between all groups (p<0.005) as shown in FIG. 55. Normalized residence time values increased from approximately 10° to 10² as stenosis severity increased from 25% to 75%, with significant differences between groups (p<0.005) as demonstrated in FIG. 56.

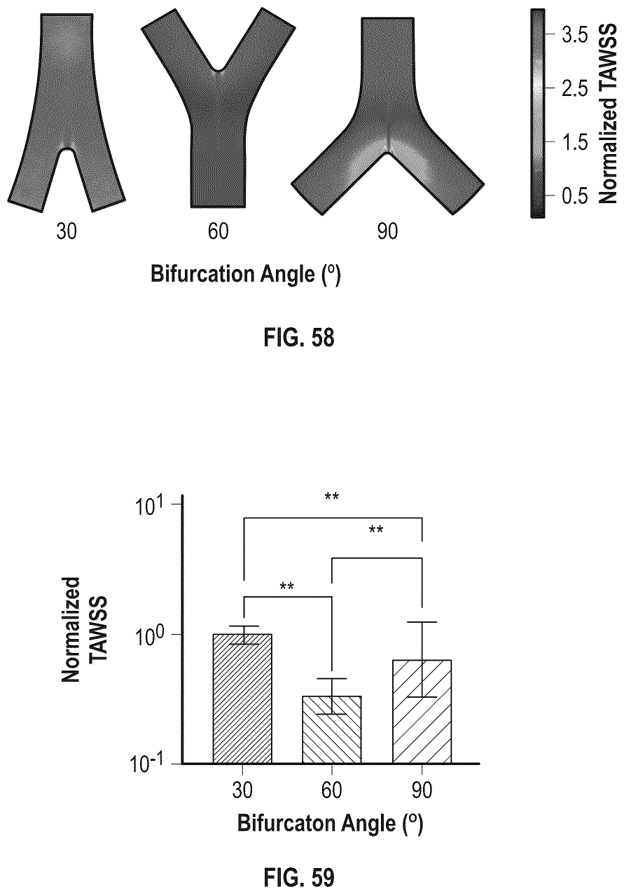

Upon RBC perfusion in bifurcation-on-chip structures, it was observed that the adhered ECM of the bifurcation-chips supported fluid flow and that the flow is evenly divided among the two post-bifurcation sections, as shown in FIG. 57. The computational analysis on bifurcation-chips revealed that as the angle of bifurcation increased, the region around the bifurcation witnessed higher TAWSS values, as shown in FIG. 58, as wider angles imply the presence of more fluidic resistance and hence fluid flow disruption may be enhanced. TAWSS values increased from approximately 0.5 to 3.5 (normalized) as bifurcation angles increased from 30° to 90°, with significant differences observed between angle groups (p<0.005) as shown in FIG. 59.

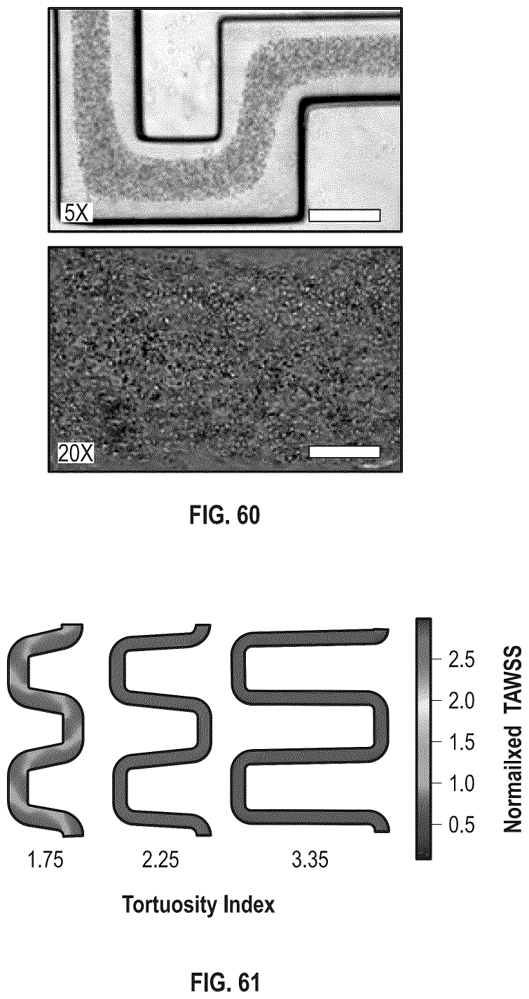

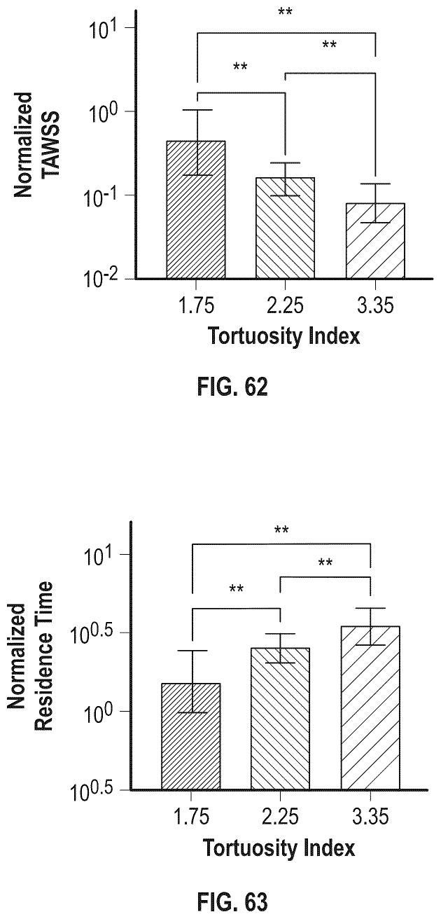

Finally, RBCs were also perfused through the tortuous vessels to confirm that the embedded ECM layer can support perfusion and to validate the effect of tortuous architecture on flow. Slower movement of cells in the outermost corners of the lumen was observed, as shown in FIG. 60. This was further supported through computational fluid dynamics (CFD) studies where the outermost corners of vessels had lower TAWSS levels across all variations; however the severity of reduction diminished as the tortuosity increased, as shown in FIG. 61. Longer tortuous channels also resulted in an overall lower average TAWSS as compared to vessels with a shorter flow path, as shown in FIG. 62. The CFD studies confirm the clinically observed linkage between severe tortuosity and resulting low shear stress, which is associated with a higher risk of disease development and progression. TAWSS values ranged from approximately 0.5 to 2.5 (normalized) across different tortuosity indices, with significant differences between groups (p<0.005). Normalized residence time values ranged from 10⁻¹ to 10¹, showing significant differences between tortuosity conditions (p<0.005) as demonstrated in FIG. 63.

In summary, by regulating and controlling the gravitational lumen patterning (GLP) method, spatially intricate 3D vascular models were engineered. It was shown that the shape and size of a patterned lumen can be modulated with the structure of their external microfluidic channels. Although some representative structures and their variations were shown, this procedure can be extended to model pathological structures for a wide range of vessels observed in human circulation. These engineered vessels with various architectures can serve as platforms to investigate vascular complications strongly associated with the structure of vessels such as aneurysm, fibromuscular dysplasia, and atherosclerosis.

The following are additional embodiments of the disclosed subject matter.

A 1^{st} embodiment is a method for fabricating an in vitro vessel, the method comprising: (a) forming a substrate that defines a microfluidic passage therein extending along a longitudinal direction of extension and defined by an inner surface, wherein the microfluidic passage includes variations in size and shape along a length of the microfluidic passage; (b) positioning the substrate in a vertical orientation whereby an acute angle is formed between the longitudinal direction of extension of the microfluidic passage and a direction of gravity; and (c) culturing a plurality of first cells in the microfluidic passage while the substrate is disposed in the vertical orientation whereby an annular layer of the plurality of first cells is formed in the microfluidic passage, wherein the layer of the plurality of first cells defines a lumen extending longitudinally through the microfluidic passage, wherein the lumen comprises a complex vascular architecture comprising a bifurcation, tortuosity, a stenosis, an aneurysm, or combinations thereof.

A 2^{nd} embodiment is the method of embodiment 1, wherein the plurality of first cells comprise lymphatic endothelial cells (LECs), vascular endothelial cells (VECs), lymphatic muscle cells (LMCs), vascular muscle cells (VMCs), human umbilical vein endothelial cells (HUVECs), pericytes, tissue resident immune cells, neural cells, or combinations thereof.

A 3^{rd} embodiment is the method of one of embodiments 1-2, further comprising: (d) culturing a plurality of second cells that are different from the plurality of first cells in the microfluidic passage whereby an annular layer of the plurality of second cells is formed in the microfluidic passage.

A 4^{th} embodiment is the method of embodiment 3, wherein the layer of the plurality of first cells forms an annular inner layer of the first cells in the microfluidic passage and the layer of the plurality of second cells forms an annular outer layer of the plurality of second cells in the microfluidic passage that is radially positioned between the inner layer and the inner surface of the microfluidic passage.

A 5^{th} embodiment is the method of one of embodiments 3-4, wherein culturing the plurality of second cells is performed prior to culturing the plurality of first cells.

A 6^{th} embodiment is the method of one of embodiments 1-5, wherein the lumen has an elliptical cross-section.

A 7^{th} embodiment is the method of embodiment 6, wherein the elliptical cross-section is defined by a major axis and a minor axis extending orthogonal to the major axis, and wherein a ratio of the major axis to the minor axis is between 1.1:1 and 5:1.

An 8^{th} embodiment is the method of one of embodiments 1-7, wherein the acute angle is equal to or less than 30°.

A 9^{th} embodiment is the method of one of embodiments 1-8, wherein the complex vascular architecture comprises a stenosis, and wherein the microfluidic passage includes one or more constrictions having a cross-sectional area that is from 20% to 80% of a cross-sectional area of a non-constricted portion of the microfluidic passage.

A 10^{th} embodiment is the method of one of embodiments 1-9, wherein the complex vascular architecture comprises an aneurysm, and wherein the microfluidic passage includes one or more expansions having a cross-sectional area that is from 150% to 500% of a cross-sectional area of a non-expanded portion of the microfluidic passage.

An 11^{th} embodiment is the method of one of embodiments 1-10, wherein the complex vascular architecture comprises a bifurcation, and wherein the microfluidic passage branches into two channels at an angle between 30° and 90°.

A 12^{th} embodiment is the method of one of embodiments 1-11, wherein the complex vascular architecture comprises tortuosity, and wherein the microfluidic passage exhibits peak-to-peak height variations from 1 millimeter to 5 millimeters.

A 13^{th} embodiment is the method of one of embodiments 1-12, wherein forming the substrate comprises: (i) creating a three-dimensional mold defining the microfluidic passage geometry; (ii) forming the substrate from polydimethylsiloxane (PDMS) using the mold; and (iii) bonding the substrate to a glass slide to enclose the microfluidic passage.

A 14^{th} embodiment is the method of one of embodiments 1-13, further comprising chemically pretreating the microfluidic passage with 3-aminopropyltriethoxysilane (APTES) and glutaraldehyde to enhance bonding between the substrate and an extracellular matrix.

A 15^{th} embodiment is the method of one of embodiments 1-14, wherein culturing the plurality of first cells comprises: (i) filling the microfluidic passage with a collagen solution; (ii) introducing phosphate buffered saline (PBS) to create a lumen through gravitational lumen patterning; and (iii) seeding the plurality of first cells into the formed lumen.

A 16^{th} embodiment is an in vitro vessel comprising: a substrate forming a microfluidic channel extending along a longitudinal direction of extension and defined by an inner surface, wherein the microfluidic channel extends between a fluid inlet at a first end of the microfluidic channel and a fluid outlet located at a longitudinally opposed second end of the microfluidic channel; an annular outer layer of muscle cells positioned in the microfluidic channel and extending entirely around the longitudinal direction of extension of the microfluidic channel; and an annular inner layer of endothelial cells positioned within the outer layer of muscle cells within the microfluidic channel and extending entirely around the longitudinal direction of extension of the microfluidic channel, wherein the inner layer of endothelial cells defines a lumen extending longitudinally through the microfluidic channel and in fluid communication with both the fluid inlet and the fluid outlet formed in the substrate, wherein the lumen comprises a complex vascular architecture comprising a bifurcation, tortuosity, a stenosis, an aneurysm, or combinations thereof.

A 17^{th} embodiment is the in vitro vessel of one of embodiments 16, wherein the muscle cells of the outer layer comprise lymphatic muscle cells (LMCs) and the endothelial cells of the inner layer comprise lymphatic endothelial cells (LECs).

An 18^{th} embodiment is the in vitro vessel of one of embodiments 16-17, wherein the muscle cells of the outer layer comprise vascular muscle cells (VMCs) and the endothelial cells of the inner layer comprise vascular endothelial cells (VECs).

A 19^{th} embodiment is the in vitro vessel of one of embodiments 16-18, wherein the outer layer of muscle cells is embedded in an annular extracellular matrix (ECM) positioned in the microfluidic channel and containing collagen.

A 20^{th} embodiment is the in vitro vessel of one of embodiments 16-19, wherein a majority of the muscle cells comprising the outer layer are aligned substantially perpendicular to the longitudinal direction of extension of the microfluidic channel.

A 21^{st} embodiment is the in vitro vessel of one of embodiments 16-20, wherein a majority of the endothelial cells comprising the inner layer are aligned substantially parallel to the longitudinal direction of extension of the microfluidic channel.

A 22^{nd} embodiment is the in vitro vessel of one of embodiments 16-21, wherein the lumen has an elliptical cross-section defined by a major axis and a minor axis extending orthogonal to the major axis, and wherein a ratio of the major axis to the minor axis is between 1.1:1 and 5:1.

A 23^{rd} embodiment is the in vitro vessel of one of embodiments 16-22, wherein the complex vascular architecture comprises a stenosis characterized by a constricted lumen diameter with a cross-sectional area of less than 75% of a non-stenotic region.

A 24^{th} embodiment is the in vitro vessel of one of embodiments 16-23, wherein the complex vascular architecture comprises an aneurysm characterized by a localized expansion with a cross-sectional area of at least 200% of a non-aneurysmal region.

A 25^{th} embodiment is the in vitro vessel of one of embodiments 16-24, wherein the complex vascular architecture comprises a bifurcation where the lumen branches into multiple branches at an angle between 30° and 90°.

A 26^{th} embodiment is a method for drug screening comprising: (a) providing an in vitro vessel comprising: (i) a substrate forming a microfluidic channel extending along a longitudinal direction of extension and defined by an inner surface, wherein the microfluidic channel extends between a fluid inlet at a first end of the microfluidic channel and a fluid outlet located at a longitudinally opposed second end of the microfluidic channel; (ii) an annular outer layer of muscle cells positioned in the microfluidic channel and extending entirely around the longitudinal direction of extension of the microfluidic channel; and (iii) an annular inner layer of endothelial cells positioned within the outer layer of muscle cells within the microfluidic channel and extending entirely around the longitudinal direction of extension of the microfluidic channel, wherein the inner layer of endothelial cells defines a lumen extending longitudinally through the microfluidic channel and in fluid communication with both the fluid inlet and the fluid outlet formed in the substrate, wherein the lumen comprises a bifurcation, tortuosity, a stenosis, an aneurysm, or combinations thereof; (b) introducing a test compound into the lumen of the in vitro vessel; (c) perfusing fluid through the lumen to simulate physiological flow conditions; and (d) analyzing endothelial cell responses to the test compound under the complex vascular architecture conditions.

A 27^{th} embodiment is the method of embodiment 26, wherein analyzing endothelial cell responses comprises measuring a parameter, wherein the parameter comprises cell morphology, cell orientation, barrier function, inflammatory markers, or combinations thereof.

A 28^{th} embodiment is a system for modeling vascular disease comprising an in vitro vessel comprising: (i) a substrate forming a microfluidic channel extending along a longitudinal direction of extension and defined by an inner surface, wherein the microfluidic channel extends between a fluid inlet at a first end of the microfluidic channel and a fluid outlet located at a longitudinally opposed second end of the microfluidic channel; (ii) an annular outer layer of muscle cells positioned in the microfluidic channel and extending entirely around the longitudinal direction of extension of the microfluidic channel; and (iii) an annular inner layer of endothelial cells positioned in the outer layer of muscle cells within the microfluidic channel and extending entirely around the longitudinal direction of extension of the microfluidic channel, wherein the inner layer of endothelial cells defines a lumen extending longitudinally through the microfluidic channel and in fluid communication with both the fluid inlet and the fluid outlet formed in the substrate, wherein the lumen comprises a bifurcation, tortuosity, a stenosis, an aneurysm, or combinations thereof; a fluid perfusion system configured to establish controlled flow through the lumen; and an imaging system configured to monitor cellular responses within the complex vascular architecture.

A 29^{th} embodiment is the system of embodiment 28, wherein the fluid perfusion system is configured to perfuse blood and blood components selected from the group consisting of platelets, immune cells, red blood cells, and combinations thereof.

A 30^{th} embodiment is the system of one of embodiments 28-29, wherein the fluid perfusion system is configured to perfuse test materials selected from the group consisting of drugs, molecules, toxins, chemicals, and particulates relevant to material that is transported in blood or lymphatic vessels in vivo or used in preclinical research.

A 31^{st} embodiment is the system of one of embodiments 28-30, wherein the fluid perfusion system is configured to apply time averaged wall shear stress (TAWSS) values ranging from 0.1 to 10 dynes/cm² to simulate physiological and pathological flow conditions.

A 32^{nd} embodiment is the system of one of embodiments 28-31, further comprising a computational fluid dynamics module configured to predict hemodynamic parameters within the complex vascular architecture.

A 33^{rd} embodiment is the system of embodiment 32, wherein the computational fluid dynamics module is configured to provide real-time analysis of flow patterns, shear stress distribution, and particle residence times.

A 34^{th} embodiment is the system of one of embodiments 28-33, wherein the imaging system comprises fluorescence microscopy.

A 35^{th} embodiment is the system of one of embodiments 28-34, wherein the complex vascular architecture comprises a stenosis, an aneurysm, a bifurcation, tortuosity, or combinations thereof.

While embodiments of the disclosure have been shown and described, modifications thereof can be made by one skilled in the art without departing from the scope or teachings herein. The embodiments described herein are exemplary only and are not limiting. Many variations and modifications of the systems, apparatus, and processes described herein are possible and are within the scope of the disclosure. For example, the relative dimensions of various parts, the materials from which the various parts are made, and other parameters can be varied. Accordingly, the scope of protection is not limited to the embodiments described herein, but is only limited by the claims that follow, the scope of which shall include all equivalents of the subject matter of the claims. Unless expressly stated otherwise, the steps in a method claim may be performed in any order. The recitation of identifiers such as (a), (b), (c) or (1), (2), (3) before steps in a method claim are not intended to and do not specify a particular order to the steps, but rather are used to simplify subsequent reference to such steps.

What is claimed is:

1. A method for fabricating an in vitro vessel, the method comprising:
(a) forming a substrate that defines a microfluidic passage therein extending along a longitudinal direction of extension and defined by an inner surface, wherein the microfluidic passage includes variations in size and shape along a length of the microfluidic passage;
(b) positioning the substrate in a vertical orientation whereby an acute angle is formed between the longitudinal direction of extension of the microfluidic passage and a direction of gravity; and
(c) culturing a plurality of first cells in the microfluidic passage while the substrate is disposed in the vertical orientation whereby an annular layer of the plurality of first cells is formed in the microfluidic passage, wherein the layer of the plurality of first cells defines a lumen extending longitudinally through the microfluidic passage, wherein the lumen comprises a complex vascular architecture comprising a bifurcation, tortuosity, a stenosis, an aneurysm, or combinations thereof.

2. The method of claim 1, wherein the plurality of first cells comprise lymphatic endothelial cells (LECs), vascular endothelial cells (VECs), lymphatic muscle cells (LMCs), vascular muscle cells (VMCs), human umbilical vein endothelial cells (HUVECs), pericytes, tissue resident immune cells, neural cells, or combinations thereof.

3. The method of claim 1, further comprising:
(d) culturing a plurality of second cells that are different from the plurality of first cells in the microfluidic passage whereby an annular layer of the plurality of second cells is formed in the microfluidic passage.

4. The method of claim 3, wherein the layer of the plurality of first cells forms an annular inner layer of the first cells in the microfluidic passage and the layer of the plurality of second cells forms an annular outer layer of the plurality of second cells in the microfluidic passage that is radially positioned between the inner layer and the inner surface of the microfluidic passage.

5. The method of claim 3, wherein culturing the plurality of second cells is performed prior to culturing the plurality of first cells.

6. The method of claim 1, wherein the lumen has an elliptical cross-section.

7. The method of claim 6, wherein the elliptical cross-section is defined by a major axis and a minor axis extending orthogonal to the major axis, and wherein a ratio of the major axis to the minor axis is between 1.1:1 and 5:1.

8. The method of claim 1, wherein the acute angle is equal to or less than 30°.

9. The method of claim 1, wherein the complex vascular architecture comprises a stenosis, and wherein the microfluidic passage includes one or more constrictions having a cross-sectional area that is from 20% to 80% of a cross-sectional area of a non-constricted portion of the microfluidic passage.

10. The method of claim 1, wherein the complex vascular architecture comprises an aneurysm, and wherein the microfluidic passage includes one or more expansions having a cross-sectional area that is from 150% to 500% of a cross-sectional area of a non-expanded portion of the microfluidic passage.

11. The method of claim 1, wherein the complex vascular architecture comprises a bifurcation, and wherein the microfluidic passage branches into two channels at an angle between 30° and 90°.

12. The method of claim 1, wherein the complex vascular architecture comprises tortuosity, and wherein the microfluidic passage exhibits peak-to-peak height variations from 1 millimeter to 5 millimeters.

13. The method of claim 1, wherein forming the substrate comprises:
(i) creating a three-dimensional mold defining the microfluidic passage geometry;
(ii) forming the substrate from polydimethylsiloxane (PDMS) using the mold; and
(iii) bonding the substrate to a glass slide to enclose the microfluidic passage.

14. The method of claim 1, further comprising chemically pretreating the microfluidic passage with 3-aminopropyltriethoxysilane (APTES) and glutaraldehyde to enhance bonding between the substrate and an extracellular matrix.

15. The method of claim 1, wherein culturing the plurality of first cells comprises:
(i) filling the microfluidic passage with a collagen solution;
(ii) introducing phosphate buffered saline (PBS) to create a lumen through gravitational lumen patterning; and
(iii) seeding the plurality of first cells into the formed lumen.

16. An in vitro vessel comprising:
a substrate forming a microfluidic channel extending along a longitudinal direction of extension and defined by an inner surface, wherein the microfluidic channel extends between a fluid inlet at a first end of the microfluidic channel and a fluid outlet located at a longitudinally opposed second end of the microfluidic channel;
an annular outer layer of muscle cells positioned in the microfluidic channel and extending entirely around the longitudinal direction of extension of the microfluidic channel; and
an annular inner layer of endothelial cells positioned within the outer layer of muscle cells within the microfluidic channel and extending entirely around the longitudinal direction of extension of the microfluidic channel, wherein the inner layer of endothelial cells defines a lumen extending longitudinally through the microfluidic channel and in fluid communication with both the fluid inlet and the fluid outlet formed in the substrate, wherein the lumen comprises a complex vascular architecture comprising a bifurcation,
tortuosity, a stenosis, an aneurysm, or combinations thereof.

17. The in vitro vessel of claim 16, wherein the muscle cells of the outer layer comprise lymphatic muscle cells (LMCs) and the endothelial cells of the inner layer comprise lymphatic endothelial cells (LECs).

18. The in vitro vessel of claim 16, wherein the muscle cells of the outer layer comprise vascular muscle cells (VMCs) and the endothelial cells of the inner layer comprise vascular endothelial cells (VECs).

19. The in vitro vessel of claim 16, wherein the outer layer of muscle cells is embedded in an annular extracellular matrix (ECM) positioned in the microfluidic channel and containing collagen.

20. The in vitro vessel of claim 16, wherein a majority of the muscle cells comprising the outer layer are aligned substantially perpendicular to the longitudinal direction of extension of the microfluidic channel and wherein a majority of the endothelial cells comprising the inner layer are aligned substantially parallel to the longitudinal direction of extension of the microfluidic channel.
